(19) United States
(12) Patent Application Publication
(10) Pub. No.: US 2025/0375599 A1
(43) Pub. Date: Dec. 11, 2025

(54) HELICAL NOSECONE

(71) Applicant: EDWARDS LIFESCIENCES CORPORATION, Irvine, CA (US)

(72) Inventors: Peleg Harel, Haifa (IL); Eitan Atias, Tel Aviv (IL); Noa Axelrod Manela, Kadima (IL); Ofir Witzman, Harish (IL)

(73) Assignee: EDWARDS LIFESCIENCES CORPORATION, Irvine, CA (US)

(21) Appl. No.: 19/306,154

(22) Filed: Aug. 21, 2025

Related U.S. Application Data
(63) Continuation of application No. PCT/US2024/019507, filed on Mar. 12, 2024.
(60) Provisional application No. 63/451,871, filed on Mar. 13, 2023.

(51) Int. Cl. A61M 29/00; A61F 2/24; A61M 25/00

(52) U.S. Cl. CPC A61M 29/00; A61F 2/2427; A61M 25/0082

(57) ABSTRACT

The present disclosure relates to a helical nosecone that can be used to dilate a puncture formed in a target tissue, and assemblies that can include the helical nosecone and a perforating member that can be used to form the pre-dilated puncture. In an example, a helical nosecone comprises a helical slot spiraling around a tapering portion of the helical nosecone, wherein the helical slot defines a plurality of helical turns. The helical nosecone can be advanced in a screw-like motion through a puncture formed in a target tissue, such as a host leaflet of an existing valvular structure, so as to expand the puncture and form an opening.

### CROSS-REFERENCE TO RELATED APPLICATIONS

This application is a continuation of International Application No. PCT/US2024/019507, filed Mar. 12, 2024, which claims the benefit of U.S. Provisional Application No. 63/451,871, filed Mar. 13, 2023, which is incorporated by reference herein.

### FIELD

The present disclosure relates helical nosecones that can be used to dilate a puncture formed in a target tissue to create an opening within the tissue, to assemblies that include such helical nosecones and methods for utilized such assemblies, for example to modify existing valvular structures (such as leaflets of a native heart valve or previously-implanted prosthetic valve) prior to implantation of a guest prosthetic heart valve, utilizing tissue perforation assemblies that include a helical nosecone.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, method that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

According to some aspects of the disclosure, there is provided a tissue perforation assembly, comprising a helical nosecone which defines a nosecone channel. The helical nosecone comprises a tapering portion extending from a nosecone distal end to a tapering portion proximal end and a helical slot spiraling around the tapering portion. The helical slot defines a plurality of helical turns.

In one of its basic configurations, a delivery assembly comprises a guest prosthetic valve and a delivery apparatus comprising a tissue perforation assembly. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the guest prosthetic valve can comprise a frame movable between a radially compressed and a radially expanded configuration.

In some examples, the delivery apparatus can comprise a handle.

In some examples, the delivery apparatus can comprise a balloon catheter optionally extending from the handle.

In some examples, the delivery apparatus can comprise a balloon optionally mounted on the balloon catheter.

In some examples, the balloon catheter optionally defines a balloon catheter lumen.

In some examples, the balloon mounted on the balloon catheter is optionally in fluid communication with the balloon catheter lumen.

In some examples, the balloon is optionally configured to transition between deflated and inflated states thereof.

In some examples, the tissue perforation assembly can comprise a helical nosecone optionally defining a nosecone channel.

In some examples, the helical nosecone can comprise a tapering portion extending from a nosecone distal end to a tapering portion proximal end.

In some examples, the helical nosecone can comprise a helical slot spiraling around the tapering portion.

In some examples, the helical slot can define a plurality of helical turns.

In one of its basic methods, a method of forming an opening in a target tissue comprises advancing a tissue perforation assembly to a target tissue. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the tissue perforation assembly optionally comprises a perforating member.

In some examples, the tissue perforation assembly optionally comprises a helical nosecone.

In some examples, the helical nosecone optionally defines a nosecone channel.

In some examples, the helical nosecone can comprise a helical slot optionally spiraling around a tapering portion of the helical nosecone.

In some examples, the helical slot optionally defines a plurality of helical turns.

In some examples, the method comprises forming, with the perforating member, a pilot puncture within the target tissue.

In some examples, the method comprises advancing the helical nosecone in a screw-like motion through the pilot puncture.

In one of its basic methods, a method of implanting-a guest prosthetic valve within a host valvular structure comprises advancing a delivery assembly that comprises a delivery apparatus carrying a guest prosthetic valve in a radially compressed state, to a host valvular structure. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the delivery apparatus can comprise a balloon optionally mounted on a balloon catheter.

In some examples, the tissue perforation assembly optionally comprises a perforating member.

In some examples, the tissue perforation assembly optionally comprises a helical nosecone.

In some examples, the helical nosecone optionally defines a nosecone channel.

In some examples, the helical nosecone can comprise a helical slot optionally spiraling around a tapering portion of the helical nosecone.

In some examples, the helical slot optionally defines a plurality of helical turns.

In some examples, the method further forming, with the perforating member, a pilot puncture within a host leaflet of the host valvular structure.

In some examples, the method comprises advancing the helical nosecone, optionally in a screw-like motion, through the pilot puncture.

In some examples, the method can comprise positioning the balloon in a deflated state thereof, optionally along with the guest prosthetic valve disposed in a compressed state over the balloon, within the pilot puncture.

In some examples, the method optionally comprises inflating the balloon so as to radially expand the guest prosthetic valve.

The method and configurations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4 shows a view in perspective of an exemplary tissue perforation assembly that includes a helical nosecone.

FIG. 5 shows a cross sectional view of the tissue perforation assembly of FIG. 4.

FIG. 6 is a cross sectional view of an exemplary tissue perforation assembly, shown with a guidewire passing through the helical nosecone.

FIG. 7 is a cross sectional view of an exemplary tissue perforation assembly, shown with a tubular perforating member passing through the helical nosecone.

FIG. 8A is a simplified side view of a tissue perforation assembly positioned with the helical nosecone proximal to a host leaflet, according to an example.

FIG. 8B is a simplified side view of the tissue perforation cutting of FIG. 8A with a tubular perforating member passed through the host leaflet.

FIG. 8C is a simplified side view of the tissue perforation assembly of FIG. 8A with the helical nosecone positioned in the host leaflet.

FIG. 9 shows a delivery assembly comprising a delivery apparatus that carries a prosthetic valve, and includes a tissue perforation assembly.

FIG. 10 is a cross-sectional view of the delivery apparatus of FIG. 9.

FIG. 11A shows the delivery assembly of FIG. 9 with a distal portion of the helical nosecone passed through a pilot puncture or a slightly expanded leaflet opening of the host leaflet.

FIG. 11B shows the delivery assembly of FIG. 9 with most of the helical nosecone passed through the host leaflet.

FIG. 12A is a simplified side view of the delivery assembly of FIG. 9 with a guest prosthetic valve crimped proximal to a deflated balloon which is positioned proximal to the leaflet opening.

FIG. 12B is a simplified side view of the delivery assembly of FIG. 12A with the guest prosthetic valve positioned over the deflated balloon, proximal to the leaflet opening.

FIG. 12C is a simplified side view of the delivery assembly of FIG. 12A with the compressed guest prosthetic valve and the deflated balloon positioned inside the leaflet opening of the host leaflet.

FIG. 12D is a simplified side view of the delivery assembly of FIG. 12A with the guest prosthetic valve expanded by the inflated balloon inside the host valvular structure.

FIG. 13 shows a previously implanted prosthetic valve subsequent to forming the leaflet opening in a host leaflet thereof.

FIG. 14 shows a configuration in which a guest prosthetic valve has been expanded within the leaflet opening of a host prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

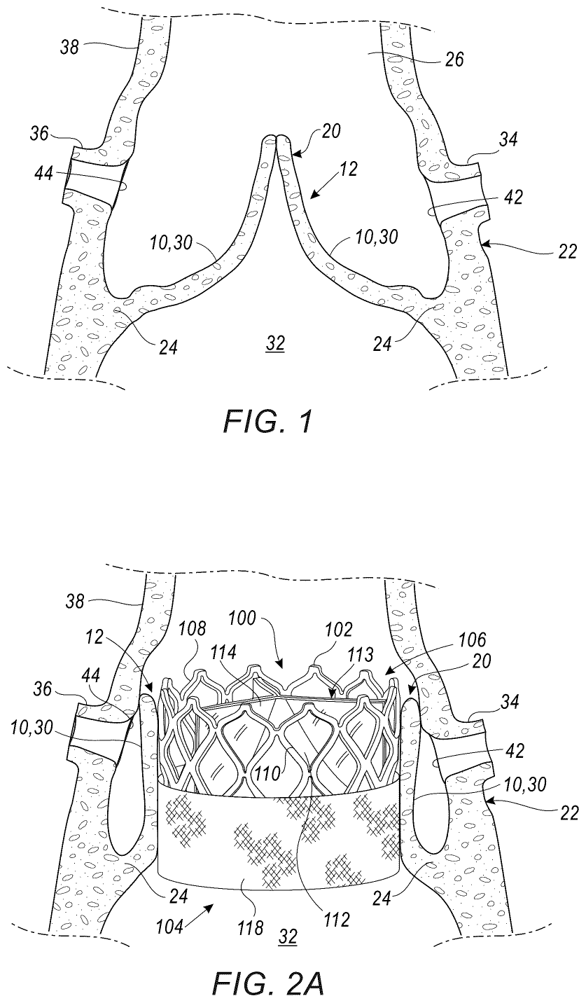

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

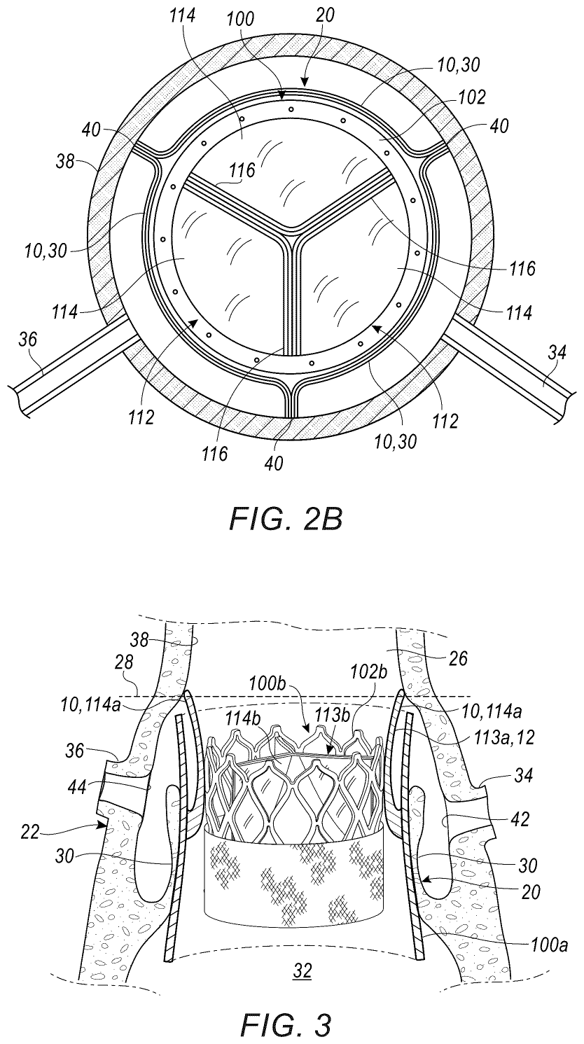

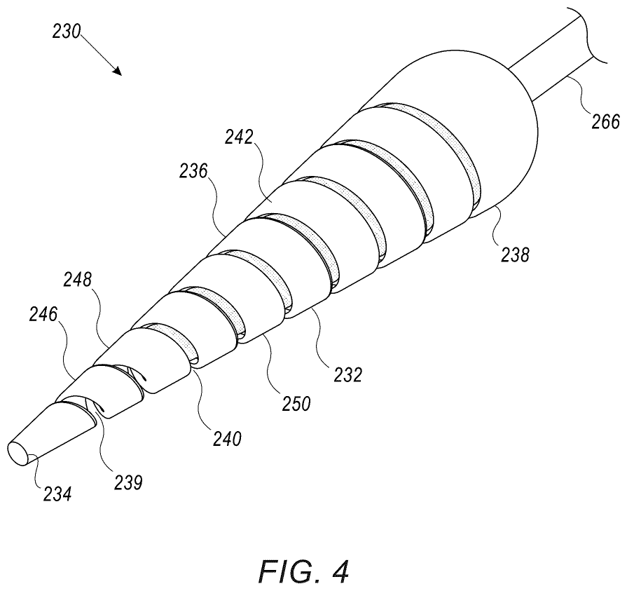

FIGS. 2A-2B show an exemplary prosthetic valve 100 that can optionally be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can optionally be crimped on or retained by an implant delivery apparatus (such as delivery apparatus 202 described below with respect to FIG. 4, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can optionally be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can optionally be made of various suitable materials, including plastically-deformable materials such as, but not limited to, stainless steel, a nickel-based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can optionally be made of shape-memory materials such as, but not limited to, nickel-titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 can optionally be an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can optionally include a plurality of angled struts and vertical or axial struts. At least some of the struts 108 can be pivotable or bendable relative to each other, so as to permit frame expansion or compression. For example, the frame 102 can optionally be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can optionally include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can optionally be arranged together to form prosthetic valve commissures 116 that are coupled (directly or indirectly) to respective portions of the frame 102, thereby securing at least a portion of the valvular structure 113 to the frame 102. The prosthetic valve leaflets 114 can optionally be made from, in whole or part, biological material (for example, pericardium), biocompatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can optionally comprise at least one skirt or sealing member. For example, the prosthetic valve 100 can optionally include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can optionally be sutured to the inner skirt along a scalloped line (not shown). An inner skirt can optionally be coupled to the frame 102 via sutures or another form of coupler.

The prosthetic valve 100 can optionally comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can optionally be formed of a single sheet of material that extends continuously around the inner surface of frame 102. In some cases, the outer skirt 118 can optionally be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can optionally be a surgically implanted prosthetic heart valve (known as a “surgical valve”) or a transcatheter heart valve. The term “guest valve”, as used herein, refers to a prosthetic valve implanted in a host valve, which can optionally be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and optionally implant a guest prosthetic valve 100 within the modified valvular structure 12, optionally without the need to switch between separate delivery apparatuses for each function.

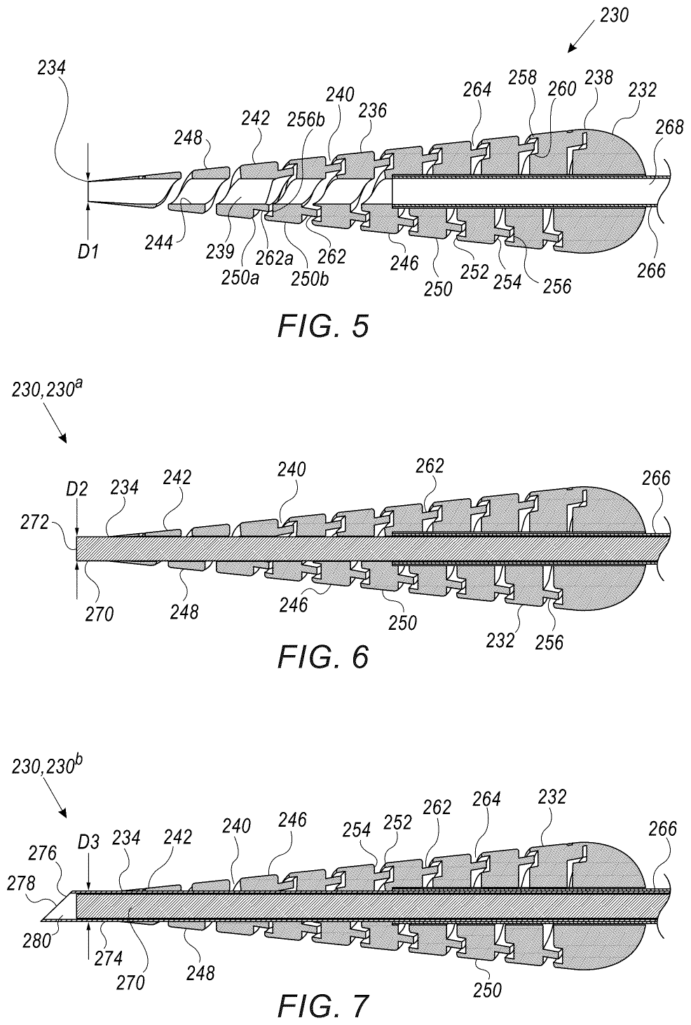

FIGS. 4-7 show exemplary tissue perforation assemblies 230 that can optionally be used to perforate and/or cut a tissue, such as a host leaflet 10 of a host valvular structure 12, and to dilate the perforation or cut to expand the opening formed within the tissue. Tissue perforation assembly 230 comprises a helical nosecone 232 defining a nosecone channel 239. FIG. 4 shows a view in perspective of a tissue perforation assembly 230. FIG. 5 shows a cross-sectional view of the tissue perforation assembly 230 of FIG. 4. The helical nosecone 232 includes a helical slot 240 and spiraling along a tapering portion 236 of the nosecone 232, the tapering portion 236 extending from a narrower diameter of a nosecone distal end 234 to a wider diameter of a tapering portion proximal end 238. The helical slot 240 defines a plurality of helical turns 246 which can include one or more distal turns 248 extending proximally from the nosecone distal end 234, and a plurality of proximal turns 250 extending proximally from the one or more distal turn 248. The helical slot 240 can optionally radially extend through the entire thickness of the nosecone 232, from the exterior surface enveloping the tapering portion 236 to the nosecone channel 239.

Each helical turn 246 includes an inner surface 244 facing the nosecone channel 239, and an outer surface 242 facing away from the nosecone channel 239. The inner surfaces 244 of the helical turns 246 together form at least part of the inner surface of the nosecone 232 or an outer surface of the nosecone channel 239. The outer surfaces 242 of the helical turns 246 together form at least part of the outer surface of the nosecone 232 or the outer surface of the tapering portion 236. Thus, the outer surfaces 242 of the helical turns 246 can optionally be angled, in the axial direction, relative to the corresponding inner surfaces 244, resulting in an increased thickness of each helical turn 246 between a first axial surface 252 thereof and a second axial surface 254 thereof.

The first axial surface 252 of a helical turn 246 can be defined as a surface extending between the inner 244 and outer 242 surfaces, facing the distal direction. The second axial surface 254 of a helical turn 246 can be defined as a surface extending between the inner 244 and outer 242 surfaces, facing the proximal direction. The first axial surface 252 can optionally be parallel to the second axial surface 254. The first axial surface 252 and the second axial surface 254 can optionally be generally orthogonal to the inner surface 244. While reference is made to a plurality of helical turns 246 herein, it is to be understood that adjacent helical turn 246 can be visually separated from each other by a helical slot 240 when viewed in an axially extending cross-section of the nosecone, yet that all helical turns 246, including distal turn(s) 248 and proximal turns 250, are continuous with each other, together forming a helical winding which is generally continuous and uninterrupted along the length of the helical slot 240. continuous helical extending wall of the helical nosecone 232.

A proximal turn 250 can optionally include an axial extension 262 proximally extending from its second axial surface 254. A proximal turn 250 can optionally include an axial recess 256 formed in its first axial surface 252. The axial recess 256 extends between an outer sidewall 258 which is closer to the outer surface 242, and an inner sidewall 260 which is farther from the outer surface 242 and closer to the inner surface 244. The axial extension 262 of a distal turn 248 extends toward and into the corresponding recess 256 of another distal turn positioned proximal to the axial extension. For example, FIG. 5 illustrates an axial extension 262a of a proximal turn 250a, extending proximally toward and into the axial recess 256b of a proximal turn 250b positioned proximal to the turn 250a.

The axial extension 262 is free ended, such that it terminates at a free end or tip which is not attached to the axial recess 256 it extends into. While the axial extensions 262 are illustrated to proximally extend from second axial surfaces 254, toward and into axial recesses 256 formed in first axial surfaces 252, it is to be understood that this geometry can optionally be reversed, and that in some examples, the proximal turns can optionally extend distally prom the first axial surfaces, toward and into axial recesses formed in the second axial surfaces.

The axial extension 262 extends axially through the corresponding portion of the helical slot 240, and is positioned radially inward relative to the outer surface 242 of the proximal turn 250 it extends from. The portion of the slot 240 bound between adjacent proximal turns 250, radially outward from the axial extension 262, forms an outer gap 264 between the adjacent proximal turns 250. Stated otherwise, the outer gap 264 is defined as the space formed between adjacent proximal turns 250, radially bound between the axial extension 262 and an outer surface of the tapering portion 236 that is coextensive with an envelope representing the outermost shape of the tapering portion 236. For example, an outer surface of the tapering portion 236 illustrated in FIGS. 4-5 refers to the portions of the outer surface of nosecone 232 that forms conical or frusto-conical surface.

In contrast to the proximal turns 250, a distal turn 248 can optionally be devoid of an axial extension and/or an axial recess. As shown in FIG. 5, this results in the external environment surrounding the helical nosecone 232 to be exposed to the nosecone channel 239 through the portions of the helical slot 240 extending between adjacent distal turns 248, while the axial extensions 262 prevents the surrounding environment from being directly exposed to the nosecone channel 239 between adjacent proximal turns 250. In some examples, the outer gap 264 can optionally define a gap width, which is the axial distance between the axial surfaces 252, 254 on both sides of the outer gap 264, and the axial extension 262 can optionally have a length which is greater than the gap width, to extend past the gap width and into the corresponding axial recess 256. When viewed from the outer side of the nosecone 232, the axial extension 262 can optionally block the line of sight toward the nosecone channel 239, beyond the axial extensions 262. In some examples, the axial extension 262 can optionally angularly extend from the corresponding axial surface. For example, the axial extension 262 can optionally be parallel to the outer surface 242 of the proximal turn 250 it extends from.

The depth of outer gap 264 can be defined as the radial distance between the axial extension 262 and the outer surface of the tapering portion 236 that is coextensive with an envelope representing the outermost shape of the tapering portion 236. In some examples, all outer gaps 264 have substantially equal depths. In some examples, the depths of all outer gaps 264 are within a range of 20% of each other. Such configuration can result in radial distances between the nosecone channel 239 and the axial extension 262 that increase in size in the proximal direction. The depth of the helical slot 240 at any portion thereof between adjacent distal turns 248 can be defined as the radial distance between the nosecone channel 239 and the outer surface of the tapering portion 236 that is coextensive with an envelope representing the outermost shape of the tapering portion 236. In some examples, the helical slot 240 defines an unequal depth between adjacent distal turns 248, wherein the depth of the helical slot 240 can optionally increase in the proximal direction. In some examples, the depth of some or all outer gaps 264 is substantially equal to the maximal depth value of the helical slot 240. In some examples, the depth of some or all outer gaps 264 is within a range of 20% of the maximal depth value of the helical slot 240.

The nosecone channel 239 defines an opening of the helical nosecone 232 at the nosecone distal end 234. The nosecone distal end 234 can define an outer diameter D1.

A nosecone shaft 266 defining a nosecone shaft lumen 268 can optionally be coupled to the helical nosecone 232 and extend proximally therefrom. The nosecone channel 239 can optionally be continuous with the nosecone shaft lumen 268. The nosecone shaft 266 can optionally be coupled, directly or indirectly, to the helical nosecone 232. In some examples, a distal portion of the nosecone shaft 266 can be coupled to a proximal portion of the helical nosecone 232, as illustrated in FIG. 5. Attachment of helical nosecone 232 to a distal portion of nosecone shaft 266 can be achieved by a variety of methods, such as overmolding, radio-frequency welding, through an adhesive, and/or a combination thereof. In some examples (not illustrated), the nosecone shaft 266 can optionally extend through the entire length of the nosecone channel 239, such that a distal end of the nosecone shaft 266 is aligned with the nosecone distal end 234 and defines the distal opening of the helical nosecone. In some examples (not illustrated), the nosecone shaft 266 is coupled to one or more components, such as collars or other connectors, which are in turn attached to the helical nosecone 232.

The helical slot 240 can advantageously result in increased flexibility of nosecone 232, to assist in advancement of the tissue perforation assembly 230 through the patient's vasculature. Flexibility of the nosecone 232 can assist in navigating around a bent or curve in the patient's vasculature. Even though the nosecone 232 can be less flexible at a proximal portion thereof due to increase in thickness of the tapering portion 236, increased flexibility of the thinner distal portion of the nosecone 232 allows a certain length thereof to get around a bend in a patient's vasculature, and create a “follow-the-leader” effect with the remainder of the nosecone 232.

The tissue perforation assembly 230 can further comprise a perforating member, configured to axially extend through nosecone channel 239 and/or nosecone shaft lumen 268 268, and pierce through a target tissue, such as a host leaflet 10 of a host valvular structure 12. FIG. 5 illustrates a tissue perforation assembly 230 without showing a perforating member.

Various exemplary implementations for tissue perforation assemblies 230 can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, tissue perforation assembly 230ª is an exemplary implementation of tissue perforation assembly 230, and thus includes all of the features described for tissue perforation assembly 230 throughout the current disclosure, except that while a tissue perforation assembly 230 can include any type of perforating member, tissue perforation assembly 230ª is shown to include a guidewire 270 that can serve as a perforating member, without inclusion of an additional perforating member therearound, as will be described in further detail below.

FIG. 6 shows a cross-section view of tissue perforation assembly 230a. As mentioned above, tissue perforation assembly 230ª is an exemplary implementation of tissue perforation assembly 230, and thus includes all of the features described for tissue perforation assembly 230 throughout the current disclosure, except that a perforating guidewire 270 can extend through the nosecone channel 239 of tissue perforation assembly 230ª, without including any other tubular perforation member passable through the nosecone channel 239.

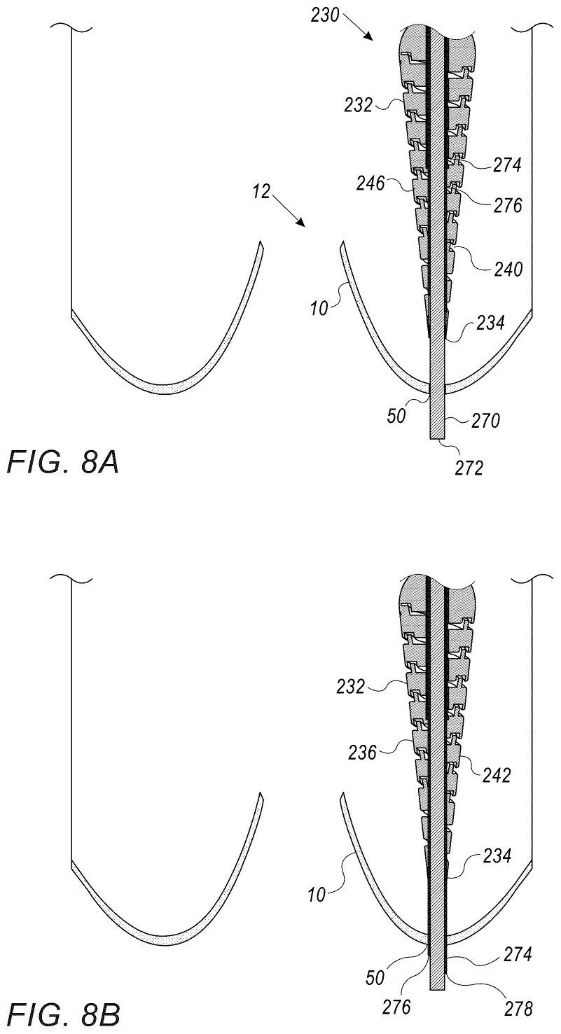

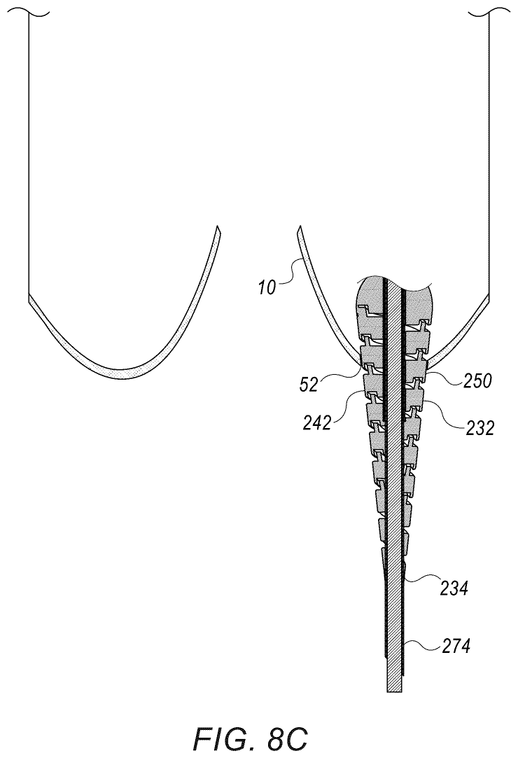

As mentioned, in some examples, the guidewire 270 can optionally be used as a perforating or lacerating member for forming a pilot puncture 50 (shown in FIG. 8A, for example). In such examples, the guidewire 270 can optionally be a relatively stiff wire having a distal tip 272 configured to pierce the host leaflet 10 when the guidewire 270 is pressed against the leaflet. In some examples, the guidewire 270 can optionally include a radio-frequency (RF) energy delivery tip 272 to assist with penetration through the leaflet tissue. For this purpose, a suitable RF energy device may optionally be coupled to the guidewire 270, and the RF energy device can apply the RF energy to the guidewire tip 272 to penetrate the host leaflet 10. In any examples disclosed herein wherein a guidewire is used to puncture a target tissue, such as a host leaflet, the guidewire 270 can optionally be coupled to a source of RF energy that applies RF energy to the tip of the guidewire.

The guidewire 270 can define a guidewire diameter D2, and terminate at a guidewire tip 272. The nosecone channel 239 and/or nosecone shaft lumen 268 of tissue perforation assembly 230ª can optionally be sized to allow passage of the guidewire 270 therethrough. In some examples, nosecone shaft 266 and/or nosecone channel 239 has an inner liner or layer formed of Teflon® to minimize sliding friction with the guidewire 270. Such a liner can be added to the inner surfaces 244 of at least some of the helical turns 246.

In some examples, the outer diameter D1 of nosecone distal end 234 is substantially equal to the guidewire diameter D2. In some examples, the diameter D1 is not greater than 120% of the diameter D2. In some examples, the diameter D1 is not greater than 110% of the diameter D2. A diameter D1 that approximates guidewire diameter D2 can result in a relatively smooth transition between the guidewire 270 and the outer surface of the nosecone 232, such that when the guidewire 270 pierces a target tissue, such as a host leaflet 10, further advancement of the nosecone 232 into the pilot puncture will allow the tissue surrounding the pilot puncture to climb over the outer surface of nosecone 232, such that nosecone 232 can expand the opening as it is pushed further therethrough.

FIG. 7 shows a cross-section view of a tissue perforation assembly 230b. Tissue perforation assembly 230b is an exemplary implementation of tissue perforation assembly 230, and thus includes all of the features described for tissue perforation assembly 230 throughout the current disclosure, except that the tissue perforation assembly 230b further comprises a tubular perforating member 274 extending through the nosecone channel 239 and/or the nosecone shaft lumen 268. In some examples, the tubular perforating member 274 is axially movable relative to the helical nosecone 232 and/or nosecone shaft 266. The tubular perforating member 274 can optionally define a perforating member lumen 280 sized to allow axial passage of guidewire 270 therethrough.

Perforating member 274 comprises a distal end portion 276 configured to pierce a target tissue, such as a host leaflet 10 of a host valvular structure 12, to form a pilot puncture 50 in the target tissue when a distal end portion 276 is positioned distal to the nosecone distal end 234. In some examples, the distal end portion 276 of perforating member 274 is configured to be selectively translated in the proximal or distal directions relative to the helical nosecone 232.

In some examples, the perforating member 274 may optionally include and/or be a needle, such as a spring-loaded needle and/or a Veress needle. As shown in FIG. 7, the distal end portion 276 of the perforating member 274 can optionally terminate at an angled surface 278. The angled surface 278 can optionally have a sharp cutting edge to facilitate piercing the host leaflet 10 (or any other target tissue) when the needle is pressed against the leaflet.

In some examples, the guidewire 270 extending through the tubular perforating member 274 is a lacerating guidewire that can optionally be used in combination with the tubular perforating member 274, such that guidewire 270 can be utilized to form an initial puncture 50 in the leaflet 10, after which the perforating member 274 can optionally be advanced through the host leaflet 10 to form a slightly larger pilot puncture for subsequent advancement of the helical nosecone 232 through the host leaflet 10.

In some examples, the guidewire tip 272 is not necessarily sharp enough or otherwise configured to puncture through the host leaflet 10, in which case the guidewire 270 can optionally be utilized for advancement of the tissue perforation assembly 230 toward the valvular structure 12, but terminate in proximity of the host leaflet 10 without piercing through it, and perforating member 274 can optionally be then advanced toward and into the host leaflet 10, to form the pilot puncture 50.

The tubular perforating member 274 can define an outer diameter D3, which can optionally be uniform along its length. The nosecone channel 239 and/or nosecone shaft lumen 268 of tissue perforation assembly 230b can optionally be sized to allow passage of the tubular perforating member 274 therethrough. In some examples, nosecone shaft 266 and/or nosecone channel 239 has an inner liner or layer formed of Teflon® to minimize sliding friction with tubular perforating member 274. Such a liner can be added to the inner surfaces 244 of at least some of the helical turns 246. In some examples, tubular perforating member 274 has an inner liner or layer formed of Teflon® to minimize sliding friction with the guidewire 270.

In some examples, the outer diameter D1 of nosecone distal end 234 is substantially equal to the outer diameter D3 of tubular perforating member 274. In some examples, the diameter D1 is not greater than 120% of the diameter D3. In some examples, the diameter D1 is not greater than 110% of the diameter D3. A diameter D1 that approximates outer diameter D3 of the tubular perforating member 274 can result in a relatively smooth transition between the perforating member 274 and the outer surface of the nosecone 232, such that when the perforating member 274 pierces a target tissue, such as a host leaflet 10, further advancement of the nosecone 232 into the pilot puncture will allow the tissue surrounding the pilot puncture to climb over the outer surface of nosecone 232, such that nosecone 232 can expand the opening as it is pushed further therethrough.

The generally conical shape of nosecone 232, and its tapering portion 236, can facilitate forming the leaflet opening 52 in the host leaflet 10 (or a tissue opening in another type of target tissue). Moreover, the helical slot 240 formed around helical nosecone 232 can allow the helical nosecone 232 to be rotated during advancement through the host leaflet 10, resulting in a screw-like motion of the helical nosecone 232 through the leaflet 10 to facilitate easier passage of the nosecone 232 through the host leaflet 10, with lower resistance of the tissue to the nosecone's gradually increasing diameter, due to the screw-motion that allows the nosecone 232 to be “screwed” through the tissue. For example, advancing the nosecone 232 through the host leaflet (for example, through a pilot puncture 50 formed in the host leaflet, such as by the guidewire 270 and/or perforating member 274) while rotating it around its axis of symmetry, can force the leaflet opening to expand in diameter with the increasing diameter of the tapering portion 236 of nosecone 232, as the helical nosecone 232 is gradually “screwed” through the host leaflet 10.

During insertion and advancement of the helical nosecone 232 through the host leaflet 10, such as in a “screw” motion as described above, the inherent resiliency of the host leaflet 10 may urge the leaflet 10 radially inwardly against the nosecone 232. When a portion of the helical slot 240 passes through the opening in the host leaflet 10, the leaflet 10 thus extends into the slot 240. When passing along portions of the slot 240 disposed between distal turns 248, the leaflet 10 extends through the slot 240 and contacts the component positioned inside the nosecone channel 239 at this axial position, such as the guidewire 270, the tubular perforating member 274, and/or the nosecone shaft 266. As the helical nosecone 232 continues advancement in a “screw”-like motion through the leaflet 10, a relevant portion of the leaflet 10 climbs from the slot 240 over the outer surface 242 of the subsequent helical turn 246, and then extends into the next portion of the helical slot 240 between the subsequent adjacent helical turns 246.

When passing along portions of the slot 240 disposed between proximal turns 248, the leaflet 10 extends through the outer gap 264, which is the portion of the slot 240 radially outward to the axial extensions 262, and contacts the corresponding axial extension 262, without reaching all the way toward the nosecone channel 239. As the helical nosecone 232 continues advancement in a “screw”-like motion through the leaflet 10, a relevant portion of the leaflet 10 can climb from outer gap 264 over the outer surface 242 of the subsequent proximal turn 250, and then extend into the next portion of the outer gap 264 between the subsequent adjacent proximal turns 250, or it can climb over the outer surface of the nosecone channel, such as at or proximate the tapering portion proximal end 238, after passing the most proximal end of helical slot 240 through the leaflet 10.

When the helical turns 246 comprise one or more distal turns 248, the radial depth of the portions of the slot 240 between adjacent distal turns 248 can optionally be relatively small, such that the host leaflet 10 can easily climb from a position inside slot 240 to the outer surface of the subsequent helical turn 246 as the helical nosecone 232 is “screwed” through the host leaflet 10. As the thickness of the nosecone 232 increases in the proximal direction, the depth of a slot 240 from an outer surface of the nosecone to the nosecone channel 239 is increased, such that when the depth is too large, and absent of axial extensions 262, the leaflet 10 would have extended deeper into the slot 240 at such regions, making it harder for the leaflet to climb over to the outer surface of the subsequent helical turn.

Advantageously, forming the axial extensions 262 can solve this challenge as the axial extensions 262 prevent the tissue from extending all the way toward the nosecone channel 239. Instead, the tissue of the host leaflet extends through the outer gap 264 to a limited radial distance, equal to the depth of the outer gap, and is supported by the axial extension 262 which is positioned closer to the outer surface 242 of the subsequent proximal turn 250, making it easier for the leaflet to climb thereover during advancement of the nosecone 232 through the leaflet 10.

While the helical nosecone 232 in the illustrated examples is shown to include both distal turn 248 devoid of axial extensions, and proximal turns 250 with axial extensions 262, in some examples, the helical turns 246 can optionally include only proximal turns 250 equipped with axial extensions 262, and no distal turns devoid of axial extensions. This can optionally be implemented for helical nosecones 232 with a helical slot 240 that forms a slot depth which is too high at its distal-most end, such that it may be preferable to include axial extensions 262 in all helical turns. In some examples, the helical turns 246 can optionally include only distal turns 248 devoid of axial extensions, such that none of the helical turns include a helical extension. This can be implemented for helical nosecones 232 with a relatively shallow helical slot 240 along its entire length, that does not necessitate inclusion of such axial extensions to support the tissue of the host leaflet 10.

While the helical slot 240 between proximal turns 250 is shown to extend all the way toward the nosecone channel 239, so as to define a gap or space that extends between the axial extension 262 and the nosecone channel 239, it is to be understood that in some examples, a helical slot 240 can optionally extend along at least some of its length to a depth that does not extend all the way to the nosecone channel, but is rather formed with a shallower radial depth that can be uniform along some or all of its length, and can optionally be similar to the depth shown for outer gaps 264. In such cases, the nosecone includes full-matter between the nosecone channel 239 and the radial floor of the helical slot, meaning that no axial extension or axial recess needs to be defined along any of the helical turns, as the shallower depth of the helical slot, at least between regions corresponding to the proximal turns, serves as the shallower support from which the tissue of the leaflet can climb over the outer surface of a subsequent turn (examples not shown). The illustrated examples, in which the helical slot 240 does extends through the entire thickness of the helical nosecone 232, may be advantageous over this configuration, as the fully-extending slot 240 as illustrated imparts increased flexibility of the helical nosecone 232, to facilitate navigating the helical nosecone 232 through the patient's vasculature.

FIGS. 8A-8C illustrate some steps in a method for utilizing a tissue perforating assembly 230 for forming an opening within a target tissue. An exemplary implementation of the method is illustrated in FIGS. 8A-8C with respect to forming a leaflet hole inside a host leaflet, which can optionally be performed prior to implanting a guest prosthetic valve inside the host valvular structure. The tissue perforating assembly 230 can optionally be used to perforate, cut, and/or tear a host leaflet 10, such as a native leaflet 30 or a prosthetic valve leaflet 114 of a previously implanted prosthetic valve. While tissue perforating assembly 230b is illustrated throughout FIGS. 8A-8C, it is to be understood that other implementations of tissue perforating assemblies 230 described in the current specification can optionally be used in a similar manner.

The distal end portion of the tissue perforating assembly 230, including helical nosecone 232, is configured to be inserted into a patient's vasculature, such as within an ascending aorta, and to be advanced towards the host leaflet 10, wherein the guidewire 270 can optionally pierce through the host leaflet 10 as shown in FIG. 8A. Positioning tissue perforating assembly 230 relative to the host leaflet 10 may optionally comprise advancing the tissue perforating assembly 230 toward the leaflet via guidewire 270. For example, the guidewire 270 can optionally be inserted into the patient's vasculature, and then tissue perforating assembly 230 may optionally be advanced toward the host leaflet 10 over the guidewire 270.

For a tissue perforation assembly 230 that includes a tubular perforating member 274, delivery of the tissue perforating assembly 230 toward the target tissue, such as host leaflet 10 of host valvular structure 12, can optionally be performed such that the distal end portion 276 of the perforating member 274 is concealed inside the nosecone 232 and/or nosecone shaft 266, to avoid damage that may be caused to internal anatomical structures of the patient's body due to accidental contact with an optionally sharp distal end portion 276. As mentioned above, a perforating member 274, when provided, is configured to be selectively translated in the proximal or distal directions relative to helical nosecone 232. In some examples, the nosecone shaft 266 and the tubular perforating member 274 are configured to be movable axially relative to each other in the proximal and distal directions. Thus, distal advancement of the tubular perforating member 274 serves to expose its sharp distal end portion 276 from the helical nosecone, extending past the nosecone distal end 234.

The tubular perforating member 274, when provided, is configured to puncture and/or cut through the host leaflet 10 to form a pilot puncture 50 within host leaflet 10. Thus, the distal end portion 276 can optionally be pushed toward and through the host leaflet 10 to form the pilot puncture 50 as shown in FIG. 8B, after which it can optionally be retracted back to re-conceal the distal end portion 276, for example inside the nosecone channel 239 and/or nosecone shaft lumen 268.

As mentioned, in some examples, the guidewire 270 can optionally be used as a perforating or lacerating member for forming a pilot puncture 50 (as shown in FIG. 8A, for example), such as by including a relatively sharp guidewire tip 272 that can be forcibly pushed through the host leaflet 10 to puncture the tissue, and/or including an RF energy delivery tip 272. When the guidewire 270 is used to pierce the leaflet 10, the tissue perforation assembly 230 can be devoid of a tubular perforating member 274, or it can also include a tubular perforating member 274 used in combination with the guidewire 270 that forms an initial puncture in the leaflet 10. For example, the guidewire 270 can optionally be used to form an initial pilot puncture 50 (see FIG. 8A), after which the tubular perforating member 274 can optionally be advanced through the leaflet to form a slightly larger pilot puncture (see FIG. 8B) for subsequent advancement of the helical nosecone 232 through the host leaflet 10.

In some examples, the guidewire 270 is used as a perforating member without any additional separate perforating member, such as a tubular perforating member 274, disposed thereover, such that the guidewire 270 can optionally be utilized as the sole component that forms the pilot puncture 50, allowing the helical nosecone 232 to pass through the pilot puncture 50, in a manner similar to that illustrated in FIG. 8C, but without the tubular perforating member 274.

In some examples, the guidewire 270 is used as a perforating member that can optionally be used in addition to a tubular perforating member 274, such that the guidewire 270 can form an initial puncture via a sharp tip 272 or an RF energy delivery tip 272, as illustrated in FIG. 8A, followed by penetration of the tubular perforating member 274 into the leaflet 10 to form the pilot puncture 50, or a pilot puncture 50 which is greater in size than an initial puncture formed by the guidewire tip 272, as shown in FIG. 8B.

In some examples, the guidewire tip 272 is not necessarily sharp enough or otherwise configured to puncture through the host leaflet 10, in which case the guidewire 270 can optionally be utilized for advancement of the tissue perforation assembly 230 toward the valvular structure 12, but terminate in proximity of the host leaflet 10 without piercing through it (for example, remaining above host leaflet 10 instead of passing through the tissue as shown in FIG. 8A), and the distal end portion 276 of tubular perforating member 274 can optionally be then advanced toward and into the host leaflet 10, to form the pilot puncture 50 in a similar manner to that illustrated in FIG. 8B.

Subsequent to forming the pilot puncture 50, and as shown in FIG. 8C, the helical nosecone 232 can optionally be inserted into the pilot puncture 50 and advanced in a screw-like motions to expand the pilot puncture 50 to form a leaflet opening 52. In some examples, the distal end portion 276 of tubular perforating member 274 can optionally be kept in position, such as below the host leaflet 10, while the helical nosecone 232 is rotatably advanced through the leaflet opening 52, until the helical nosecone 232 fully covers the distal end portion 276, concealing its sharp edges from the surrounding tissues. In some examples, the distal end portion 276 can optionally be retracted proximally back into the nosecone 232 after formation of the leaflet opening 52, and prior to advancement of the helical nosecone 232 through the leaflet opening 52.

In some examples, passing the helical nosecone 232 through the host leaflet 10 may cause the host leaflet 10 to rip and/or tear such that the leaflet opening 52 is not a bounded hole. Stated differently, in such examples, the leaflet opening 52 may be formed by a tear that extends to the free edge of the host leaflet 10 (the coaptation edge of the leaflet).

The tissue perforation assembly 230 may be configured to form the leaflet opening 52 in any of a variety of host valvular structures 12. In the example of FIGS. 8A-8C, the host valvular structure 12 can optionally be the valvular structure 113 of a previously implanted prosthetic valve, such as the prosthetic valve 100a of FIG. 3. In such examples, using the tissue perforation assembly 230 as described herein to form the leaflet opening 52 in a previously implanted prosthetic valve may optionally be followed by steps for implanting a guest prosthetic valve 100b within the previously implanted prosthetic valve 100a (for example, via a ViV procedure).

Similarly, the host valvular structure 12 in the example of FIGS. 8A-8C can optionally be a valvular structure 29 of a native heart valve, such as the native aortic valve 20 shown in FIGS. 2A-2B. In such examples, the tissue perforation assembly 230 can be configured to puncture a native leaflet 30 of the native aortic valve 20. In other examples, the host valvular structure and/or the native valve may refer to another valve of a patient's heart, such as a mitral valve, a pulmonary valve, or a tricuspid valve.

While illustrated and described above with respect to forming a leaflet opening 52 within a host leaflet 10, it is to be understood that the tissue perforation assembly 230 may be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some implementations, a tissue perforation assembly 230 may be utilized to form an opening through the interatrial septum, for example at the site of the fossa ovalis, which is a region of the septum containing tissue of lesser thickness than is typical of the rest of the septum. Thus, any example of tissue perforation assembly 230 described herein can optionally be utilized in a manner similar to that described with respect to FIGS. 8A-8C, to form a tissue opening, equivalent to leaflet opening 52 described with respect to FIG. 8A-8C, in a target tissue, equivalent to a host leaflet 10 described with respect to FIGS. 8A-8C.

In some examples, tissue perforation assembly 230 can optionally be part of a delivery assembly that includes a delivery apparatus carrying a prosthetic valve. Any delivery assembly disclosed herein, comprises a delivery apparatus that can include a tissue perforation assembly according to any of the examples described above, and a balloon expandable prosthetic valve. While examples of a delivery assembly described in the current disclosure, are shown to include an exemplary delivery apparatus and a balloon expandable prosthetic valve, it should be understood that a delivery apparatus that includes a tissue cutting assembly according to any example of the current disclosure can optionally be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

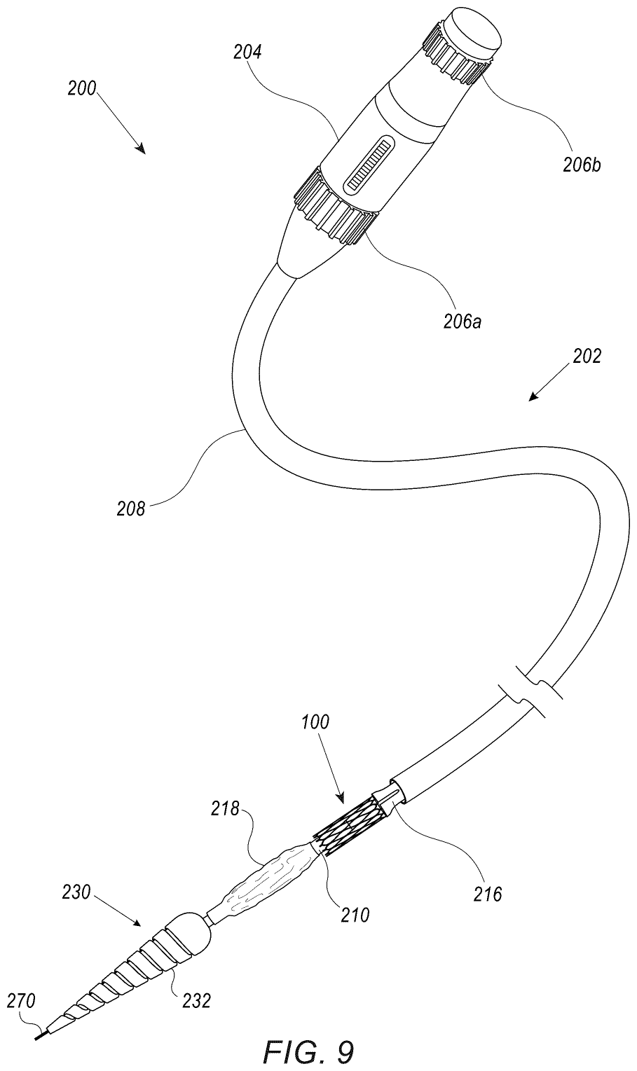

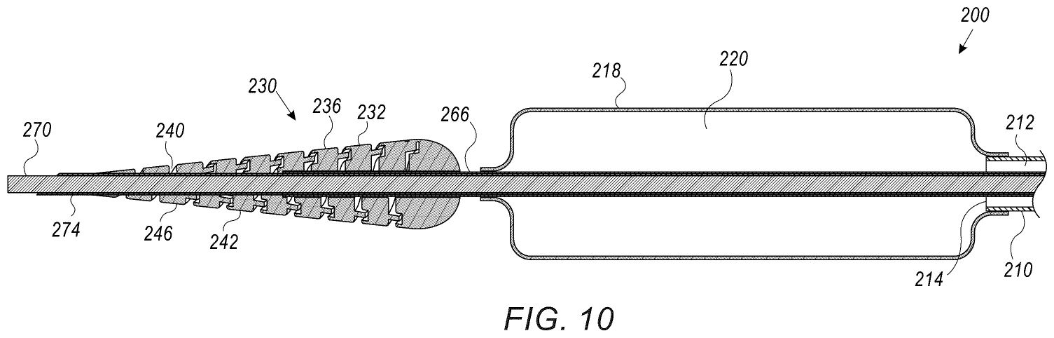

FIG. 9 illustrates an exemplary delivery assembly 200 that includes an exemplary delivery apparatus 202 adapted to deliver a balloon expandable prosthetic valve 100, such as prosthetic valve 100 described above with respect to FIGS. 2A-2B. FIG. 10 shows a cross-sectional view of a distal portion of delivery apparatus 202 comprising a tissue perforation assembly 230. According to some examples, the delivery apparatus 202 further includes a handle 204 and a balloon catheter 210 having a balloon 218 mounted on its distal end, proximal to the helical nosecone 232. The balloon expandable prosthetic valve 100 can optionally be carried in a crimped state over the balloon catheter 210. Optionally, an outer delivery shaft 208 can concentrically extend over the balloon catheter 210, and a push shaft 216 can be disposed over the balloon catheter 210, optionally between the balloon catheter 210 and the delivery shaft 208. The nosecone shaft 266 can optionally extend through the lumen 212 of the balloon catheter 210 and an internal cavity 220 of the balloon 218 towards the handle 204.

The outer delivery shaft 208, the push shaft 216, the balloon catheter 210, the nosecone shaft 266, and when provided, the tubular perforation member 274, can optionally be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic valve 100 from the outer delivery shaft 208.

The proximal ends of the balloon catheter 210, the outer delivery shaft 208, the push shaft 216, and the nosecone shaft 266, can optionally be coupled to the handle 204. During delivery of the prosthetic valve 100, the handle 204 can optionally be maneuvered by an operator (for example, a clinician or a surgeon) to axially advance or retract components of the delivery apparatus 202, such as the guidewire 270, the nosecone shaft 266, the balloon catheter 210, the outer delivery shaft 208, push shaft 216, and/or the tubular perforation member 274, through the patient's vasculature and/or along the target site of implantation, as well as to inflate the balloon 218 mounted on the balloon catheter 210 so as to expand the prosthetic valve 100, and to deflate the balloon 218 and retract the delivery apparatus 202 once the guest prosthetic valve 100 is mounted in the implantation site (for example, within the host valve).

The balloon catheter 210 can extend through the handle 204 and optionally be fluidly connectable to a fluid source for inflating the balloon 218. The fluid source comprises an inflation fluid. The term “inflation fluid”, as used herein, means a fluid (for example, saline, though other liquids or gas can be used) used for inflating the balloon 218. An inflation fluid source is in fluid communication with the balloon catheter lumen 212, such as the annular space between the inner surface of balloon catheter 210 and the outer surface of nosecone shaft 266 extending therethrough, such that fluid from the fluid source can optionally flow through the balloon catheter lumen 212, and into the balloon 218 to inflate it.

The handle 204 can optionally include a steering mechanism configured to adjust the curvature of the distal end portion of the delivery apparatus 202. In the illustrated example, the handle 204 can optionally include an adjustment member, such as the illustrated rotatable knob 206a, which in turn is operatively coupled to the proximal end portion of a pull wire. The pull wire can optionally extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can optionally further include an adjustment mechanism including an adjustment member, such as the illustrated rotatable knob 206b. The adjustment mechanism can be configured to adjust the axial position of the push shaft 216 relative to the outer balloon catheter. The handle can optionally include additional adjustment mechanisms controllable by additional knobs to maneuver additional components of the delivery apparatus 202, such as axial movement of the guidewire 270, axial movement of the nosecone shaft 266, axial movement of the balloon catheter 210, axial movement of a push shaft 216, and/or axial movement of the tubular perforation member 274, relative to other shafts, wires or components of the delivery apparatus 202.

The prosthetic valve 100 can optionally be carried by the delivery apparatus 202 during delivery in a crimped state, and expanded by inflation of balloon 218 to secure it in a native heart valve annulus (such as aortic annulus 24) or against a previously implanted prosthetic valve. In an exemplary implantation procedure, the prosthetic valve 100 can optionally be initially crimped over the balloon catheter 210, proximal to the balloon 218. Because prosthetic valve 100 is crimped at a location different from the location of balloon 218, prosthetic valve 100 can be crimped to a lower profile than would be possible if it was crimped on top of balloon 218. This lower profile permits the clinician to more easily navigate the delivery assembly 200 (including crimped prosthetic valve 100) through a patient's vasculature to the treatment location. The lower profile of the crimped prosthetic valve is helpful when navigating through portions of the patient's vasculature which are narrow, such as the iliac artery.

When reaching the host valve, the delivery apparatus 202 can be utilized to modify at least one host leaflet 10 described above with respect to FIGS. 8A-8C, after which the deflated balloon 218, carrying crimped prosthetic valve 100 thereover, can be advanced to the host valvular structure 12 to expand the guest prosthetic valve 100. Prior to inflation of balloon 218, the push shaft 216 can optionally be advanced distally, allowing its distal end portion to contact and push against the outflow end 106 of prosthetic valve 100, pushing the prosthetic valve 100 distally therewith. The distal end of push shaft 216 is dimensioned to engage with the outflow end 106 of prosthetic valve 100 in a crimped configuration of the valve. In some implementations, the distal end portion of the push shaft 216 can optionally be flared radially outward, to terminate at a wider-diameter that can contact the prosthetic valve 100 in its crimped state. Optionally, push shaft 216 can then be advanced distally, pushing the prosthetic valve 100 therewith, until the crimped prosthetic valve 100 is disposed around the balloon 218, after which the balloon 218 can optionally be inflated to radially expand the prosthetic valve 100. Once the prosthetic valve 100 is expanded to its functional diameter within a native annulus or within a previously implanted host prosthetic valve, the balloon 218 can optionally be deflated and the delivery apparatus 202 can optionally be retrieved from the patient's body.

In some examples, the delivery assembly 200 can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can optionally be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Nosecone shaft 266, balloon catheter 210, and optional outer delivery shaft 208, can optionally be formed from any of various suitable materials, such as nylon, braided stainless steel wires, or a polyether block amide (commercially available as Pebax®). In some examples, nosecone shaft 266, balloon catheter 210, and optional outer delivery shaft 208, can optionally have longitudinal sections formed from different materials in order to vary the flexibility of the shafts along their lengths.

The nosecone shaft 266 can optionally be sized such that an annular space is formed within balloon catheter lumen 212 between balloon catheter 210 and nosecone shaft 266 along the length of balloon catheter 210. This annular space is optionally in fluid communication with one or more balloon catheter openings 214 exposed to an internal cavity 220 of the balloon 218, which can optionally be in fluid communication with a fluid source (for example, a syringe or a pump) that can optionally inject an inflation fluid (for example, saline) into balloon cavity 220. In this way, fluid from the fluid source can optionally flow through balloon catheter lumen 212, and into balloon cavity 220 via balloon catheter opening(s) 214, which serves to inflate the balloon 218 and expand and deploy a prosthetic valve 100 disposed thereon. The pressure of the inflation fluid within balloon cavity 220 may provide the force that allows the balloon 218 to expand a prosthetic valve 100 disposed thereon. Further, the balloon catheter lumen 212 may optionally be configured to withdraw fluid from balloon cavity 220 through balloon catheter opening(s) 214, to deflate the balloon 218.

While balloon catheter 210 is shown to terminate at a proximal end of balloon 218 in FIG. 10, in some examples, balloon catheter 210 can optionally extend farther in the distal direction (examples not illustrated), through a portion or through the entire length of balloon cavity 220, and one or more balloon catheter opening(s) 214 can b optionally e formed on the sidewall of balloon catheter 210, exposed laterally to balloon cavity 220.

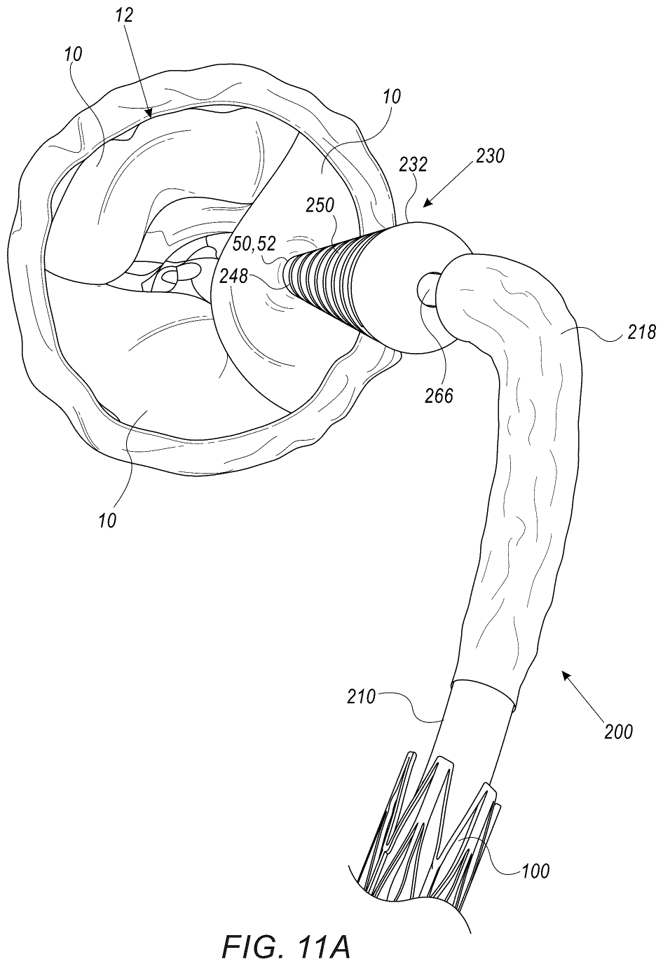

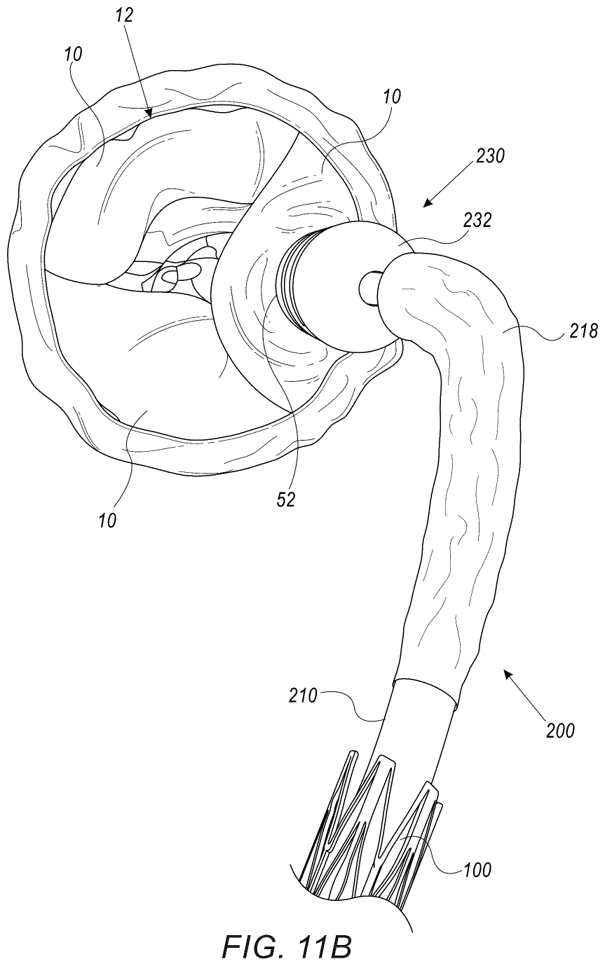

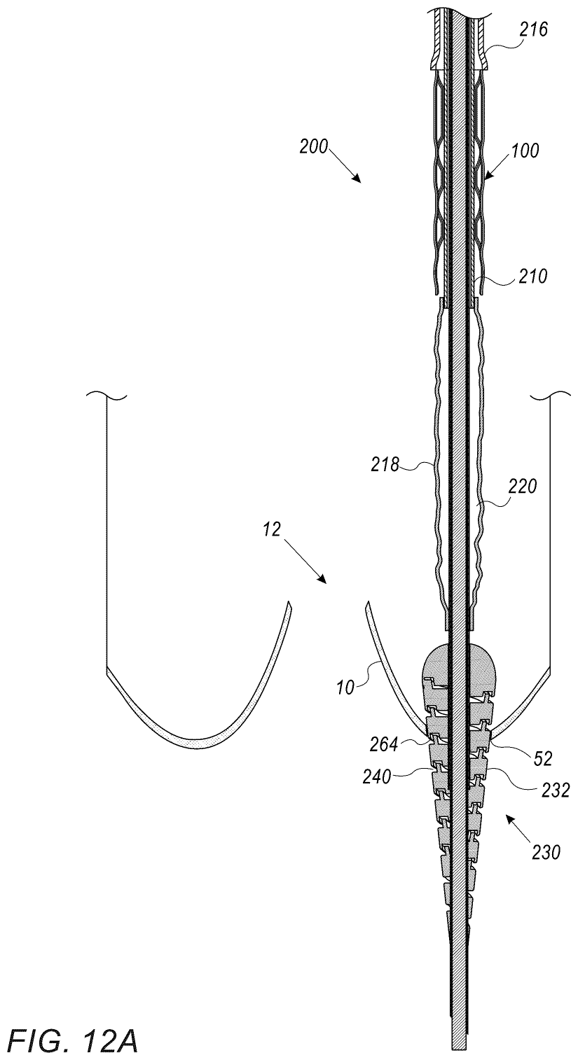

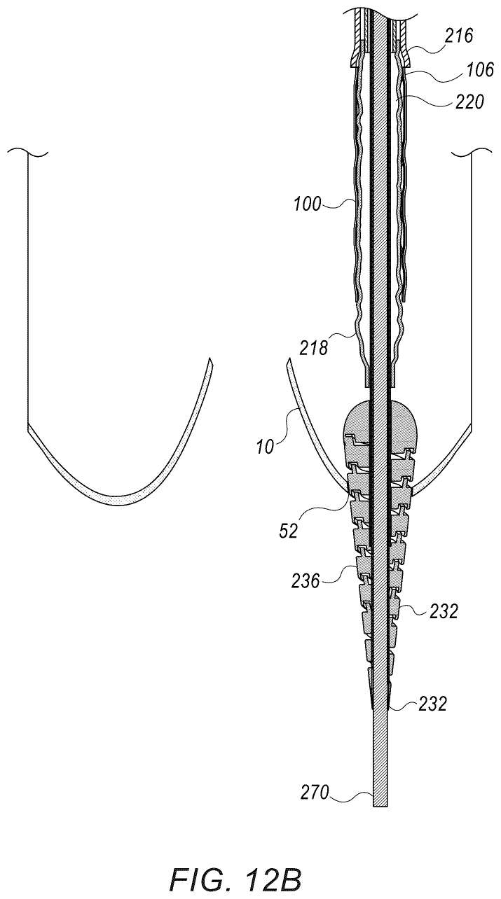

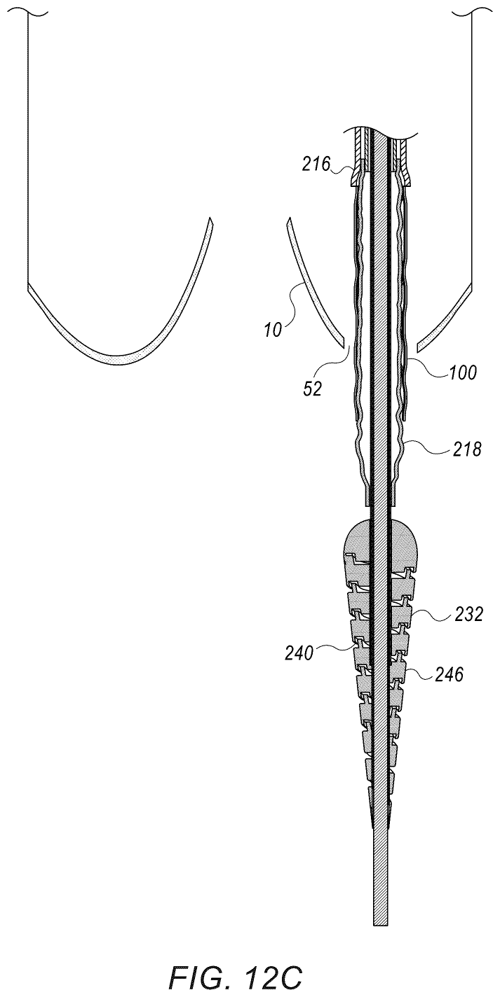

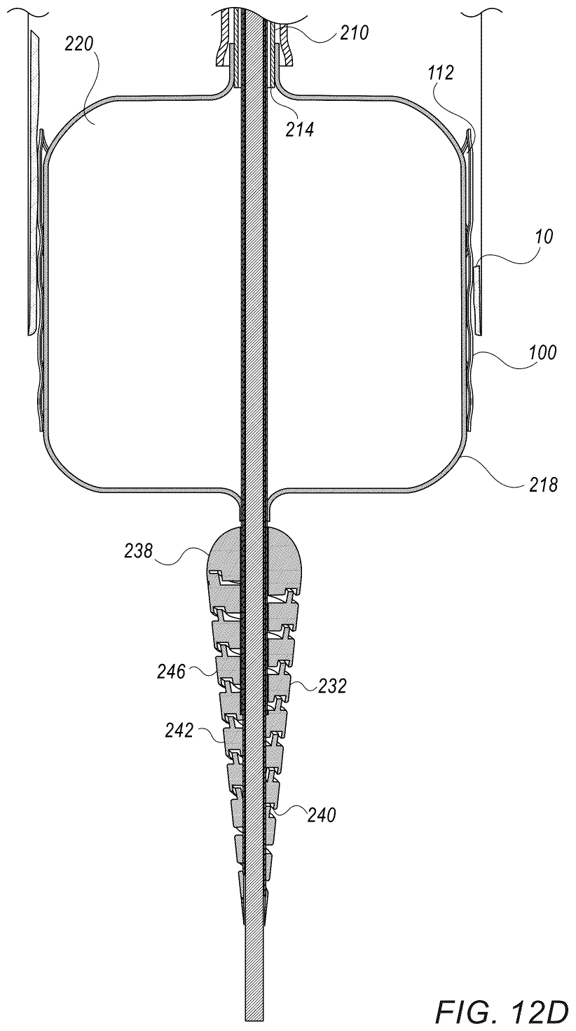

FIGS. 11A-12D show stages in an exemplary implantation procedure for implanting the prosthetic valve 100 within a host leaflet structure 12 using a transfemoral delivery procedure. In some examples, various other delivery procedures can optionally be used, such as transventricular, transapical, transseptal, etc. Delivery apparatus 202 may include any of a variety of features to facilitate positioning the guidewire 270, helical nosecone 232, and/or the tubular perforation member 274, relative to the host leaflet 10. For example, the nosecone shaft 266, balloon catheter 210, outer delivery shaft 208, and/or guidewire 270, may optionally be pre-formed, shaped, and/or curved so as to be directed and/or angled toward the host leaflet 10 when positioned in the vicinity of the host valvular structure 12. Furthermore, one or more shafts of delivery apparatus 202, such as outer delivery shaft 208, can optionally have a steering mechanism (for example, a pull wire and a corresponding adjustment mechanism in the handle 204) to steer or adjust its distal end.

Formation of a leaflet opening 52 in a host leaflet 10 by a tissue perforation assembly 230 of a delivery assembly 200 can be performed in the same manner described above with respect to FIGS. 8A-8C. FIGS. 11A-11B illustrate the tissue perforation assembly 230 utilized to form a leaflet opening 52, after optionally forming a pilot puncture 50, within the host leaflet 10. For example, FIG. 11A illustrates a distal portion of the helical nosecone 232 inserted in the host leaflet 10, optionally subsequent to forming a pilot puncture 50 by a guidewire 270 and/or a tubular perforating member 274, in accordance with examples described above. FIG. 11B illustrates a greater portion of the helical nosecone 232 advanced in a rotation “screw”-like motion through the leaflet 10, so as to dilate the puncture and form an enlarged leaflet opening 52, corresponding to the state described above with respect to FIG. 8C. As shown throughout FIGS. 11A-11B, the balloon 218, positioned proximal to the helical nosecone 232, remains in a deflated state throughout steps of the procedure equivalent to those described above with respect to FIGS. 8A-8C.

FIGS. 12A-12D show optionally subsequent steps of a method utilizing delivery assembly 200, following steps equivalent to those described above and illustrated in FIGS. 8A-8C. After formation of the leaflet opening 52 by tissue perforation assembly 230 as shown in FIGS. 8C and 12A, the push shaft 216 can optionally be utilized to distally advance the crimped prosthetic valve 100 toward and around balloon 218, as shown in FIG. 12B.

Optionally, the deflated balloon 218 and the prosthetic valve 100 disposed thereover can be then advanced and positioned within the leaflet opening 52, as shown in FIG. 12C. The push shaft 216 can optionally remain in position, abutting outflow end 106 of prosthetic valve 100 during advancement into and through the leaflet opening 52, to provide a counter force that resists proximal displacement of the prosthetic valve 100 during insertion into leaflet opening 52.

At this stage, delivering inflation fluid into the balloon cavity 220 allows the balloon 218 to inflate and expand the prosthetic valve 100, as shown in FIG. 12D. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 12B to be pushed distally to the position of balloon 218 after or during forming the leaflet opening 52, it is to be understood that the prosthetic valve 100 can optionally be pushed by push shaft 216 over balloon 218 at any other stage prior to advancement of the balloon 218 into the leaflet opening 52 as shown in FIG. 12C. For example, the prosthetic valve 100 can optionally be pushed over balloon 218 upon approximation to the site of implantation, such as upon reaching the region of the ascending aorta 26 even prior to forming the pilot puncture as shown in FIGS. 8A-8B. Similarly, it is to be understood that in some examples, the delivery assembly 200 can optionally be provided without a push shaft 216, and/or that the prosthetic valve 100 can optionally be crimped around the balloon 218 and delivered through the patient's vasculature in this position.

With the guest prosthetic valve 100 received within the leaflet opening 52, radially expanding the guest prosthetic valve, as shown in FIG. 12D, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

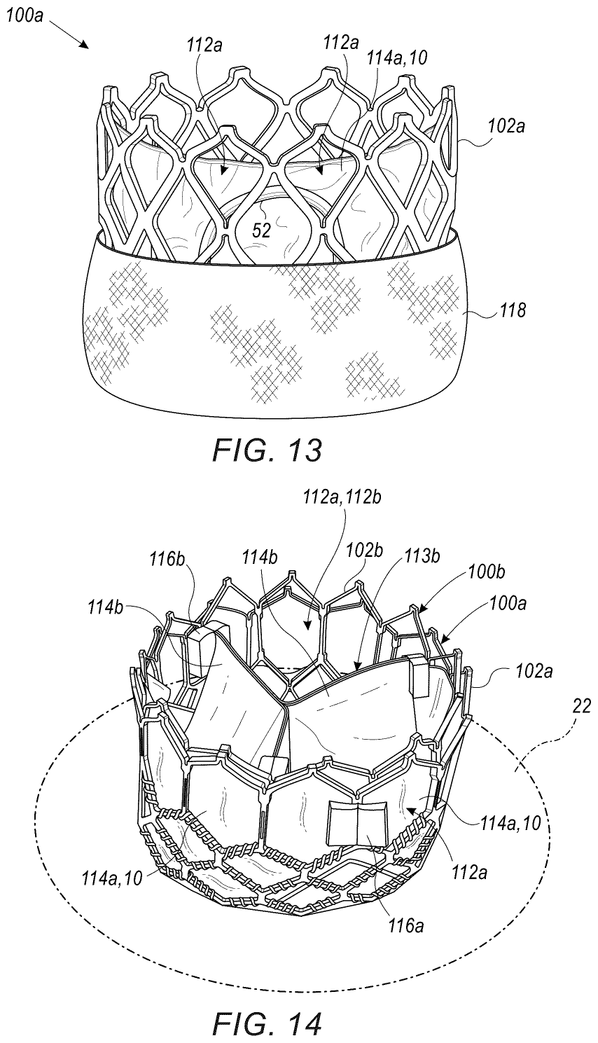

As mentioned, the delivery assemblies and methods of the current specification can optionally be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 13 shows a previously implanted prosthetic valve 100a subsequent to forming the leaflet opening 52, for example subsequent to the method described above with respect to FIGS. 8A-8C. FIG. 14 shows a configuration in which a second prosthetic valve 100b has been expanded within the leaflet opening 52 of a host prosthetic valve 100a. In the example of FIG. 14, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 13, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 14, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can optionally comprise, in some examples, repeating one or more steps disclosed throughout the current specification to form a plurality of punctures and openings in the host valvular structure. For example, steps described above with respect to FIGS. 8A-8C can be performed for forming a first leaflet opening in a first host leaflet, after which the delivery apparatus, including tissue perforation assembly 230, can optionally be retracted from the first host leaflet and steered toward another host leaflet, after which the same or equivalent steps can optionally be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries.

In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can optionally be modified such that the guest prosthetic valve can optionally be implanted by being expanded in a leaflet opening of a first host leaflet that faces the left coronary artery, and such that the second leaflet opening can optionally be formed in a second host leaflet that faces the right coronary artery (or vice-versa).

In some examples, forming the first leaflet opening can optionally be performed prior to forming the second leaflet opening. In other examples, forming the second leaflet opening can optionally be performed prior to forming the first leaflet opening. In some examples, the order of forming leaflet openings is chosen such that the final leaflet opening is formed in the host leaflet in which the guest prosthetic valve 100 is to be positioned and expanded, such as over a balloon as described above with respect to FIGS. 12A-12D.

It is to be understood that the guest prosthetic valve 100 is not limited to being implanted within an opening 52 of a leaflet. For example, in cases where the tissue perforation assembly 230 forms a full tear in a host leaflet that extends to the coaptation edge of the leaflet, the guest prosthetic valve 100 can optionally be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic heart valve can be positioned at a location between the leaflets of the host valvular structure 12 (such that the delivery assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

While a delivery assembly 200 comprising a balloon catheter 210 and a balloon-inflatable prosthetic valve 100 that can optionally be expanded by inflating a balloon 218 is described above with respect to FIGS. 9-12D, it is to be understood that utilization of other valve expansion mechanisms in combination with the tissue perforation assembly 230 are contemplated. In some examples, radial expansion of the guest prosthetic valve 100 can optionally be achieved by actuating a mechanical actuator of the guest prosthetic valve to mechanically expand a frame of the guest prosthetic valve. In some examples, the guest prosthetic valve can optionally be a self-expandable prosthetic valve that can optionally be retained during delivery toward the host valvular structure in a capsule or other restraint disposed therearound, and valve expansion can be achieved by removing the capsule or other restraint from the guest prosthetic valve to allow it to radially self-expand within the host valvular structure.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A tissue perforation assembly comprising: a helical nosecone defining a nosecone channel, the helical nosecone comprising: a tapering portion extending from a nosecone distal end to a tapering portion proximal end; and a helical slot spiraling around the tapering portion, the helical slot defining a plurality of helical turns.

Example 2. The tissue perforation assembly of any example herein, particularly example 1, further comprising a nosecone shaft attached to the helical nosecone and extending proximally therefrom, the nosecone shaft defining a nosecone shaft lumen.

Example 3. The tissue perforation assembly of any example herein, particularly example 2, wherein the nosecone shaft lumen is continuous with the nosecone channel.

Example 4. The tissue perforation assembly of any example herein, particularly any one of examples 1 to 3, wherein the helical slot radially extends through the entire thickness of the helical nosecone.

Example 5. The tissue perforation assembly of any example herein, particularly any one of examples 1 to 4, wherein each helical turn comprises an inner surface facing the nosecone channel and an outer surface facing away from the nosecone channel.

Example 6. The tissue perforation assembly of any example herein, particularly example 5, wherein the outer surface is angled, in the axial direction, relative to the inner surface.

Example 7. The tissue perforation assembly of any example herein, particularly example 5 or 6, wherein each helical turn comprises a first axial surface extending between the inner surface and the outer surface and facing the distal direction, and a second axial surface extending between the inner surface and the outer surface and facing the proximal direction.

Example 8. The tissue perforation assembly of any example herein, particularly example 7, wherein the plurality of helical turns comprises a plurality of proximal turns, wherein at least one of the plurality of proximal turns comprises an axial extension extending therefrom toward an adjacent one of the proximal turns.

Example 9. The tissue perforation assembly of any example herein, particularly example 8, wherein each of the plurality of proximal turns comprises the axial extension.

Example 10. The tissue perforation assembly of any example herein, particularly example 8 or 9, wherein at least one of the plurality of proximal turns comprises an axial recess configured to accommodate a portion of the axial extension of an adjacent proximal turn.

Example 11. The tissue perforation assembly of any example herein, particularly example 10, wherein each of the plurality of proximal turns comprises the axial recess.

Example 12. The tissue perforation assembly of any example herein, particularly any one of examples 8 to 11, wherein the axial extension is free ended.

Example 13. The tissue perforation assembly of any example herein, particularly any one of examples 8 to 12, wherein the axial extension proximally extends from the second axial surface of the corresponding proximal turn.

Example 14. The tissue perforation assembly of any example herein, particularly example 10, wherein the axial recess is formed in the first axial surface of the corresponding proximal turn.

Example 15. The tissue perforation assembly of any example herein, particularly any one of examples 8 to 12, wherein the axial extension distally extends from the first axial surface of the corresponding proximal turn.

Example 16. The tissue perforation assembly of any example herein, particularly example 10, wherein the axial recess is formed in the second axial surface of the corresponding proximal turn.

Example 17. The tissue perforation assembly of any example herein, particularly any one of examples 8 to 16, wherein the helical slot defines an outer gap between two adjacent proximal turn, radially outward to the corresponding axial extension.

Example 18. The tissue perforation assembly of any example herein, particularly any one of examples 8 to 17, wherein the axial extension is parallel to the outer surface of the corresponding proximal turn.

Example 19. The tissue perforation assembly of any example herein, particularly any one of examples 8 to 18, wherein the outer gap defines a gap depth, wherein the gap depth is less than a depth defined by the helical slot at the position of the corresponding gap.

Example 20. The tissue perforation assembly of any example herein, particularly example 19, wherein a plurality of outer gaps defined between a plurality of the proximal turns have equal gap depths.

Example 21. The tissue perforation assembly of any example herein, particularly example 19, wherein a plurality of outer gaps defined between a plurality of the proximal turns have gap depths within the range of 20% from each other.

Example 22. The tissue perforation assembly of any example herein, particularly any one of examples 8 to 21, wherein the plurality of helical turns further comprises a plurality of distal turns, distal to the proximal turns, wherein none of the plurality of distal turns comprises an axial extension extending from an axial surface thereof.

Example 23. The tissue perforation assembly of any example herein, particularly any one of examples 19 to 21, wherein the plurality of helical turns further comprises a plurality of distal turns, distal to the proximal turns, wherein none of the plurality of distal turns comprises an axial extension extending from an axial surface thereof, and wherein the gap depth is equal to or greater than a maximal depth of the helical slot defined between adjacent distal turns.

Example 24. The tissue perforation assembly of any example herein, particularly any one of examples 1 to 23, further comprising a guidewire extending through the nosecone channel.

Example 25. The tissue perforation assembly of any example herein, particularly example 24, wherein the guidewire is axially movable relative to the helical nosecone.

Example 26. The tissue perforation assembly of any example herein, particularly example 24 or 25, wherein the guidewire comprises a sharp tip configured to pierce a target tissue.

Example 27. The tissue perforation assembly of any example herein, particularly example 24 or 25, wherein the guidewire is configured to conduct RF energy of a tip thereof.

Example 28. The tissue perforation assembly of any example herein, particularly any one of examples 24 to 27, wherein an outer diameter of the nosecone distal end is not greater than 120% of a diameter defined by the guidewire.

Example 29. The tissue perforation assembly of any example herein, particularly example 28, wherein the outer diameter of the nosecone distal end is not greater than 110% of the diameter of the guidewire.

Example 30. The tissue perforation assembly of any example herein, particularly any one of examples 1 to 23, further comprising a tubular perforating member extending through the nosecone channel.

Example 31. The tissue perforation assembly of any example herein, particularly example 30, wherein the tubular perforating member is axially movable relative to the helical nosecone.

Example 32. The tissue perforation assembly of any example herein, particularly example 30 or 31, wherein the tubular perforating member defines a perforating member lumen sized to allow passage of a guidewire therethrough.

Example 33. The tissue perforation assembly of any example herein, particularly any one of examples 30 to 32, wherein the tubular perforating member comprises a distal end portion configured to pierce a target tissue.

Example 34. The tissue perforation assembly of any example herein, particularly example 33, wherein the distal end portion comprises an angled surface.

Example 35. The tissue perforation assembly of any example herein, particularly any one of examples 30 to 34, wherein an outer diameter of the nosecone distal end is not greater than 120% of an outer diameter of the tubular perforating member.

Example 36. The tissue perforation assembly of any example herein, particularly example 35, wherein the outer diameter of the nosecone distal end is not greater than 110% of the diameter of the tubular perforating member.

Example 37. The tissue perforation assembly of any example herein, particularly any one of examples 26 or 33, wherein the target tissue is a host leaflet of a host valvular structure.

Example 38. A delivery assembly comprising: a guest prosthetic valve comprising a frame movable between a radially compressed and a radially expanded configuration; and a delivery apparatus comprising: a handle; a balloon catheter extending from the handle, the balloon catheter defining a balloon catheter lumen; a balloon mounted on the balloon catheter and in fluid communication with the balloon catheter lumen, the balloon configured to transition between deflated and inflated states thereof; and a tissue perforation assembly comprising: a helical nosecone defining a nosecone channel, the helical nosecone comprising: a tapering portion extending from a nosecone distal end to a tapering portion proximal end; and a helical slot spiraling around the tapering portion, the helical slot defining a plurality of helical turns.

Example 39. The delivery assembly of any example herein, particularly example 38, wherein the helical nosecone is configured form a leaflet opening in a host leaflet of a host valvular structure by expanding a pilot formed in the host leaflet, when passed through the pilot puncture.

Example 40. The delivery assembly of any example herein, particularly example 38 or 39, wherein, when the guest prosthetic valve is disposed around the balloon and positioned within a host valvular structure, inflation of the balloon expands the guest prosthetic valve to implant the guest prosthetic valve in the host valvular structure.

Example 41. The delivery assembly of any example herein, particularly any one of examples 38 to 40, wherein the helical nosecone is configured to axially rotate around its axis when passed through the host leaflet.

Example 42. The delivery assembly of any example herein, particularly any one of examples 38 to 41, wherein the helical slot radially extends through the entire thickness of the helical nosecone.

Example 43. The delivery assembly of any example herein, particularly any one of examples 38 to 42, wherein each helical turn comprises an inner surface facing the nosecone channel and an outer surface facing away from the nosecone channel.

Example 44. The delivery assembly of any example herein, particularly example 43, wherein the outer surface is angled, in the axial direction, relative to the inner surface.

Example 45. The delivery assembly of any example herein, particularly example 43 or 44, wherein each helical turn comprises a first axial surface extending between the inner surface and the outer surface and facing the distal direction, and a second axial surface extending between the inner surface and the outer surface and facing the proximal direction.

Example 46. The delivery assembly of any example herein, particularly example 45, wherein the plurality of helical turns comprises a plurality of proximal turns, wherein at least one of the plurality of proximal turns comprises an axial extension extending therefrom toward an adjacent one of the proximal turns.

Example 47. The delivery assembly of any example herein, particularly example 46, wherein each of the plurality of proximal turns comprises the axial extension.

Example 48. The delivery assembly of any example herein, particularly example 46 or 47, wherein at least one of the plurality of proximal turns comprises an axial recess configured to accommodate a portion of the axial extension of an adjacent proximal turn.

Example 49. The delivery assembly of any example herein, particularly example 48, wherein each of the plurality of proximal turns comprises the axial recess.

Example 50. The delivery assembly of any example herein, particularly any one of examples 46 to 49, wherein the axial extension is free ended.

Example 51. The delivery assembly of any example herein, particularly any one of examples 46 to 50, wherein the axial extension proximally extends from the second axial surface of the corresponding proximal turn.

Example 52. The delivery assembly of any example herein, particularly example 49, wherein the axial recess is formed in the first axial surface of the corresponding proximal turn.

Example 53. The delivery assembly of any example herein, particularly any one of examples 46 to 50, wherein the axial extension distally extends from the first axial surface of the corresponding proximal turn.

Example 54. The delivery assembly of any example herein, particularly example 49, wherein the axial recess is formed in the second axial surface of the corresponding proximal turn.

Example 55. The delivery assembly of any example herein, particularly any one of examples 46 to 54, wherein the helical slot defines an outer gap between two adjacent proximal turn, radially outward to the corresponding axial extension.

Example 56. The delivery assembly of any example herein, particularly any one of examples 46 to 55, wherein the axial extension is parallel to the outer surface of the corresponding proximal turn.

Example 57. The delivery assembly of any example herein, particularly any one of examples 46 to 56, wherein the outer gap defines a gap depth, wherein the gap depth is less than a depth defined by the helical slot at the position of the corresponding gap.

Example 58. The delivery assembly of any example herein, particularly example 57, wherein a plurality of outer gaps defined between a plurality of the proximal turns have equal gap depths.

Example 59. The delivery assembly of any example herein, particularly example 57, wherein a plurality of outer gaps defined between a plurality of the proximal turns have gap depths within the range of 20% from each other.

Example 60. The delivery assembly of any example herein, particularly any one of examples 46 to 59, wherein the plurality of helical turns further comprises a plurality of distal turns, distal to the proximal turns, wherein none of the plurality of distal turns comprises an axial extension extending from an axial surface thereof.

Example 61. The delivery assembly of any example herein, particularly any one of examples 57 to 59, wherein the plurality of helical turns further comprises a plurality of distal turns, distal to the proximal turns, wherein none of the plurality of distal turns comprises an axial extension extending from an axial surface thereof, and wherein the gap depth is equal to or greater than a maximal depth of the helical slot defined between adjacent distal turns.

Example 62. The delivery assembly of any example herein, particularly any one of examples 46 to 61, wherein the delivery apparatus further comprises a nosecone shaft attached to the nosecone and extending proximally therefrom.

Example 63. The delivery assembly of any example herein, particularly example 62, wherein the nosecone shaft extends through the balloon catheter.

Example 64. The delivery assembly of any example herein, particularly any one of examples 46 to 63, further comprising a guidewire extending through the nosecone channel.

Example 65. The delivery assembly of any example herein, particularly example 64, the guidewire is axially movable relative to the helical nosecone.

Example 66. The delivery assembly of any example herein, particularly example 64 or 65, wherein the guidewire comprises a sharp tip configured to form a pilot puncture by piercing a host leaflet.

Example 67. The delivery assembly of any example herein, particularly example 64 or 65, further comprising an RF energy source coupled to the guidewire and configured to provide RF energy to a tip of the guidewire.

Example 68. The delivery assembly of any example herein, particularly any one of examples 46 to 63, wherein the tissue perforation assembly further comprises a tubular perforating member extending through the nosecone channel.

Example 69. The delivery assembly of any example herein, particularly example 68, wherein the tubular perforating member is axially movable relative to the helical nosecone.

Example 70. The delivery assembly of any example herein, particularly example 68 or 69, wherein the tubular perforating member comprises a distal end portion configured to form a pilot puncture by piercing a host leaflet.

Example 71. The delivery assembly of any example herein, particularly example 70, wherein the distal end portion comprises an angled surface.

Example 72. The delivery assembly of any example herein, particularly any one of examples 68 to 71, further comprising a guidewire extending through a perforating member lumen of the tubular perforating member.

Example 73. The delivery assembly of any example herein, particularly any one of examples 46 to 72, wherein the delivery apparatus further comprises a push shaft configured to push the guest prosthetic valve from a position proximal to the balloon toward the balloon.

Example 74. The delivery assembly of any example herein, particularly any one of examples 46 to 73, wherein the host valvular structure is a native valvular structure of native heart valve.

Example 75. The delivery assembly of any example herein, particularly any one of examples 46 to 73, wherein the host valvular structure is a valvular structure of previously implanted prosthetic valve that is implanted within a native heart valve.

Example 76. The delivery assembly of any example herein, particularly any one of examples 46 to 75, wherein the delivery apparatus is sterilized.

Example 77. A method of forming an opening in a target tissue, the method comprising: advancing a tissue perforation assembly that comprises a perforating member and a helical nosecone defining a nosecone channel, to a target tissue, wherein the helical nosecone comprises a helical slot spiraling around a tapering portion of the helical nosecone, the helical slot defining a plurality of helical turns; forming, with the perforating member, a pilot puncture within the target tissue; and advancing the helical nosecone in a screw-like motion through the pilot puncture.

Example 78. The method of any example herein, particularly example 77, wherein the advancing the helical nosecone through the pilot puncture is configured to expand the pilot puncture, to form a tissue opening within the target tissue.

Example 79. The method of any example herein, particularly example 77 or 78, further comprising, prior to forming the pilot puncture, positioning the helical nosecone adjacent the target tissue.

Example 80. The method of any example herein, particularly any one of examples 77 to 79, wherein the helical slot radially extends through the entire thickness of the helical nosecone.

Example 81. The method of any example herein, particularly any one of examples 77 to 80, wherein each helical turn comprises an inner surface facing the nosecone channel and an outer surface facing away from the nosecone channel.

Example 82. The method of any example herein, particularly example 81, wherein the outer surface is angled, in the axial direction, relative to the inner surface.

Example 83. The method of any example herein, particularly example 81 or 82, wherein each helical turn comprises a first axial surface extending between the inner surface and the outer surface and facing the distal direction, and a second axial surface extending between the inner surface and the outer surface and facing the proximal direction.

Example 84. The method of any example herein, particularly any one of examples 81 to 83, wherein the plurality of helical turns comprises a plurality of proximal turns, wherein at least one of the plurality of proximal turns comprises an axial extension extending therefrom toward an adjacent one of the proximal turns.

Example 85. The method of any example herein, particularly example 84, wherein at least one of the plurality of proximal turns comprises an axial recess configured to accommodate a portion of the axial extension of an adjacent proximal turn.

Example 86. The method of any example herein, particularly example 84 or 85, wherein the axial extension is free ended.

Example 87. The method of any example herein, particularly any one of examples 84 to 86, wherein the helical slot defines an outer gap between two adjacent proximal turn, radially outward to the corresponding axial extension.

Example 88. The method of any example herein, particularly any one of examples 84 to 87, wherein the axial extension is parallel to the outer surface of the corresponding proximal turn.

Example 89. The method of any example herein, particularly any one of examples 84 to 88, wherein the outer gap defines a gap depth, wherein the gap depth is less than a depth defined by the helical slot at the position of the corresponding gap.

Example 90. The method of any example herein, particularly any one of examples 84 to 89, wherein the plurality of helical turns further comprises a plurality of distal turns, distal to the proximal turns, wherein none of the plurality of distal turns comprises an axial extension extending from an axial surface thereof.

Example 91. The method of any example herein, particularly example 89, wherein the plurality of helical turns further comprises a plurality of distal turns, distal to the proximal turns, wherein none of the plurality of distal turns comprises an axial extension extending from an axial surface thereof, and wherein the gap depth is equal to or greater than a maximal depth of the helical slot defined between adjacent distal turns.

Example 92. The method of any example herein, particularly any one of examples 77 to 91, wherein the tissue perforation assembly further comprises a nosecone shaft attached to the nosecone and extending proximally therefrom.

Example 93. The method of any example herein, particularly any one of examples 77 to 92, wherein the perforating member is a guidewire extending through the nosecone channel.

Example 94. The method of any example herein, particularly example 93, wherein the forming the pilot puncture comprises applying RF energy to a tip of the guidewire.

Example 95. The method of any example herein, particularly any one of examples 77 to 92, wherein the perforating member is a tubular perforating member extending through the nosecone channel.

Example 96. The method of any example herein, particularly example 95, wherein the forming the pilot puncture comprises translating a distal end portion of the tubular perforating in a distal direction relative to the helical nosecone to pierce the target tissue to form the pilot puncture.

Example 97. The method of any example herein, particularly example 96, wherein the distal end portion comprises an angled surface.

Example 98. The method of any example herein, particularly any one of examples 77 to 92, wherein the target tissue is the interatrial septum.

Example 99. The method of any example herein, particularly example 78, wherein the target tissue is a host leaflet of a host valvular structure, and wherein the tissue opening is a leaflet opening.

Example 100. The method of any example herein, particularly example 99, wherein the host valvular structure is a native valvular structure of native heart valve.

Example 101. The method of any example herein, particularly example 99, wherein the host valvular structure is a valvular structure of previously implanted prosthetic valve that is implanted within a native heart valve.

Example 102. The method of any example herein, particularly any one of examples 99 to 101, wherein the forming the pilot puncture comprises forming a first pilot puncture in a first host leaflet, wherein the advancing the helical nosecone to form the tissue opening comprises advancing the helical nosecone through the first pilot puncture to form a first leaflet opening in the first host leaflet, and wherein, subsequent to forming the first leaflet opening, the method further comprises: retracting the tissue perforating assembly from the first host leaflet; forming, with the perforating member, a second pilot puncture within a second host leaflet; and advancing the helical nosecone in a screw-like motion through the second pilot puncture, to expand the second pilot puncture, to form a second tissue opening within the second host leaflet.

Example 103. The method of any example herein, particularly any one of examples 77 to 102, further comprising, subsequent to forming the leaflet opening: positioning a guest prosthetic valve in a radially compressed state within the host valvular structure; and radially expanding the guest prosthetic valve.

Example 104. The method of any example herein, particularly example 103, wherein the positioning the guest prosthetic valve within the host valvular structure comprises positioning the guest prosthetic valve within the leaflet opening.

Example 105. The method of any example herein, particularly example 103 or 104, wherein the radially expanding the guest prosthetic valve comprises inflating a balloon over which the guest prosthetic valve is disposed.

Example 106. The method of any example herein, particularly example 103 or 104, wherein the radially expanding the guest prosthetic valve comprises actuating a mechanical actuator of the guest prosthetic valve.

Example 107. The method of any example herein, particularly example 103 or 104, wherein the guest prosthetic valve is a self-expandable prosthetic valve, and wherein the radially expanding the guest prosthetic valve comprises removing a restraint from around the guest prosthetic valve.

Example 108. A method of implanting a guest prosthetic valve within a host valvular structure, the method comprising: advancing a delivery assembly that comprises a delivery apparatus carrying a guest prosthetic valve in a radially compressed state to a host valvular structure, wherein the delivery apparatus comprises a balloon mounted on a balloon catheter and a tissue perforation assembly which comprises a perforating member and a helical nosecone defining a nosecone channel, the helical nosecone comprising a helical slot spiraling around a tapering portion of the helical nosecone, the helical slot defining a plurality of helical turns; forming, with the perforating member, a pilot puncture within a host leaflet of the host valvular structure; advancing the helical nosecone in a screw-like motion through the pilot puncture; positioning the balloon in a deflated state thereof, along with the guest prosthetic valve disposed in a compressed state over the balloon, within the pilot puncture; and inflating the balloon so as to radially expand the guest prosthetic valve.

Example 109. The method of any example herein, particularly example 108, wherein the advancing the helical nosecone through the pilot puncture is configured to expand the pilot puncture, to form a leaflet opening within the host valvular structure.

Example 110. The method of any example herein, particularly example 108 or 109, wherein the advancing a delivery assembly to the host valvular structure comprises positioning the helical nosecone in proximity to the host leaflet.

Example 111. The method of any example herein, particularly any one of examples 108 to 110, wherein the helical slot radially extends through the entire thickness of the helical nosecone.

Example 112. The method of any example herein, particularly any one of examples 108 to 111, wherein each helical turn comprises an inner surface facing the nosecone channel and an outer surface facing away from the nosecone channel.

Example 113. The method of any example herein, particularly example 112, wherein the outer surface is angled, in the axial direction, relative to the inner surface.

Example 114. The method of any example herein, particularly example 112 or 113, wherein each helical turn comprises a first axial surface extending between the inner surface and the outer surface and facing the distal direction, and a second axial surface extending between the inner surface and the outer surface and facing the proximal direction.

Example 115. The method of any example herein, particularly any one of examples 112 to 114, wherein the plurality of helical turns comprises a plurality of proximal turns, wherein at least one of the plurality of proximal turns comprises an axial extension extending therefrom toward an adjacent one of the proximal turns.

Example 116. The method of any example herein, particularly example 115, wherein at least one of the plurality of proximal turns comprises an axial recess configured to accommodate a portion of the axial extension of an adjacent proximal turn.

Example 117. The method of any example herein, particularly example 115 or 116, wherein the axial extension is free ended.

Example 118. The method of any example herein, particularly any one of examples 115 to 117, wherein the helical slot defines an outer gap between two adjacent proximal turn, radially outward to the corresponding axial extension.

Example 119. The method of any example herein, particularly any one of examples 115 to 118, wherein the axial extension is parallel to the outer surface of the corresponding proximal turn.

Example 120. The method of any example herein, particularly any one of examples 115 to 119, wherein the outer gap defines a gap depth, wherein the gap depth is less than a depth defined by the helical slot at the position of the corresponding gap.

Example 121. The method of any example herein, particularly any one of examples 115 to 120, wherein the plurality of helical turns further comprises a plurality of distal turns, distal to the proximal turns, wherein none of the plurality of distal turns comprises an axial extension extending from an axial surface thereof.

Example 122. The method of any example herein, particularly example 120, wherein the plurality of helical turns further comprises a plurality of distal turns, distal to the proximal turns, wherein none of the plurality of distal turns comprises an axial extension extending from an axial surface thereof, and wherein the gap depth is equal to or greater than a maximal depth of the helical slot defined between adjacent distal turns.

Example 123. The method of any example herein, particularly any one of examples 108 to 122, wherein the tissue perforation assembly further comprises a nosecone shaft attached to the nosecone and extending proximally therefrom.

Example 124. The method of any example herein, particularly any one of examples 108 to 123, wherein the perforating member is a guidewire extending through the nosecone channel.

Example 125. The method of any example herein, particularly example 124, wherein the forming the pilot puncture comprises applying RF energy to a tip of the guidewire.

Example 126. The method of any example herein, particularly any one of examples 108 to 125, wherein the perforating member is a tubular perforating member extending through the nosecone channel.

Example 127. The method of any example herein, particularly example 126, wherein the forming the pilot puncture comprises translating a distal end portion of the tubular perforating in a distal direction relative to the helical nosecone to pierce the host leaflet to form the pilot puncture.

Example 128. The method of any example herein, particularly example 127, wherein the distal end portion comprises an angled surface.

Example 129. The method of any example herein, particularly any one of examples 108 to 128, wherein the delivery apparatus further comprises a nosecone shaft attached to the helical nosecone and extending proximally therefrom.

Example 130. The method of any example herein, particularly example 129, wherein the nosecone shaft extends through the balloon catheter.

Example 131. The method of any example herein, particularly any one of examples 108 to 130, wherein the positioning the balloon inside the host valvular structure comprises positioning the balloon between host leaflets of the host valvular structure.

Example 132. The method of any example herein, particularly any one of examples 108 to 130, wherein the positioning the balloon inside the host valvular structure comprises positioning the balloon inside the leaflet opening.

Example 133. The method of any example herein, particularly example 132, wherein the inflating the balloon to radially expand the guest prosthetic valve increases the size of the leaflet opening.

Example 134. The method of any example herein, particularly example 132 or 133, wherein the inflating the balloon to radially expand the guest prosthetic valve tears the host leaflet.

Example 135. The method of any example herein, particularly any one of examples 108 to 134, wherein the inflating the balloon to radially expand the guest prosthetic valve modifies the host leaflet such that the host leaflet does not obstruct a cell opening of a frame of the guest prosthetic valve.

Example 136. The method of any example herein, particularly any one of examples 108 to 135, wherein the inflating the balloon to radially expand the guest prosthetic valve moves the host leaflet to a location upstream of a downstream edge of an outer skirt of the guest prosthetic valve.

Example 137. The method of any example herein, particularly any one of examples 108 to 136, further comprising, prior to positioning the balloon inside the host valvular structure, distally pushing the guest prosthetic valve, by a push shaft of the delivery apparatus, towards and over the balloon.

Example 138. The method of any example herein, particularly example 137, wherein the positioning the balloon comprises keeping the push shaft in close proximity to a proximal end of the guest prosthetic valve, so as to provide a counterforce to prevent the guest prosthetic valve from proximally slipping from the balloon.

Example 139. The method of any example herein, particularly any one of examples 108 to 138, wherein the inflating the balloon comprises providing inflation fluid into the balloon via a lumen of the balloon catheter.

Example 140. The method of any example herein, particularly any one of examples 108 to 139, wherein the host valvular structure is a native valvular structure of native heart valve.

Example 141. The method of any example herein, particularly any one of examples 108 to 139, wherein the host valvular structure is a valvular structure of previously implanted prosthetic valve that is implanted within a native heart valve.

Example 142. The method of any example herein, particularly example 109, wherein the forming the pilot puncture comprises forming a first pilot puncture in a first host leaflet, wherein the advancing the helical nosecone to form the tissue opening comprises advancing the helical nosecone through the first pilot puncture to form a first leaflet opening in the first host leaflet, and wherein, subsequent to forming the first leaflet opening, the method further comprises: retracting the tissue perforating assembly from the first host leaflet; forming, with the perforating member, a second pilot puncture within a second host leaflet; and advancing the helical nosecone in a screw-like motion through the second pilot puncture, to expand the second pilot puncture, to form a second tissue opening within the second host leaflet.

Example 143. The method of any example herein, particularly example 142, wherein the positioning the balloon inside the host valvular structure comprises positioning the balloon inside the second leaflet opening.

Example 144. The method of any example herein, particularly any one of examples 108 to 143, further comprising, subsequent to inflating the balloon to radially expand the guest prosthetic valve, deflating the balloon and retrieving the delivery apparatus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A tissue perforation assembly, comprising:
a helical nosecone defining a nosecone channel, the helical nosecone comprising:
a tapering portion extending from a nosecone distal end to a tapering portion proximal end; and
a helical slot spiraling around the tapering portion, the helical slot defining a plurality of helical turns.

2. The tissue perforation assembly of claim 1, wherein the helical slot radially extends through the entire thickness of the helical nosecone.

3. The tissue perforation assembly of claim 1, wherein each helical turn comprises an inner surface facing the nosecone channel and an outer surface facing away from the nosecone channel.

4. The tissue perforation assembly of claim 3, wherein the outer surface is angled, in the axial direction, relative to the inner surface.

5. The tissue perforation assembly of claim 3, wherein each helical turn comprises a first axial surface extending between the inner surface and the outer surface and facing the distal direction, and a second axial surface extending between the inner surface and the outer surface and facing the proximal direction.

6. The tissue perforation assembly of claim 5, wherein the plurality of helical turns comprises a plurality of proximal turns, wherein at least one of the plurality of proximal turns comprises an axial extension extending therefrom toward an adjacent one of the proximal turns.

7. The tissue perforation assembly of claim 6, wherein each of the plurality of proximal turns comprises the axial extension.

8. The tissue perforation assembly of claim 6, wherein at least one of the plurality of proximal turns comprises an axial recess configured to accommodate a portion of the axial extension of an adjacent proximal turn.

9. The tissue perforation assembly of claim 6, wherein the helical slot defines an outer gap between two adjacent proximal turn, radially outward to the corresponding axial extension.

10. The tissue perforation assembly of claim 9, wherein the outer gap defines a gap depth, wherein the gap depth is less than a depth defined by the helical slot at the position of the corresponding gap.

11. The tissue perforation assembly of claim 10, wherein a plurality of outer gaps defined between a plurality of the proximal turns have equal gap depths.

12. The tissue perforation assembly of claim 10, wherein a plurality of outer gaps defined between a plurality of the proximal turns have gap depths within the range of 20% from each other.

13. The tissue perforation assembly of claim 1, further comprising a tubular perforating member extending through the nosecone channel.

14. The tissue perforation assembly of claim 13, wherein the tubular perforating member comprises a distal end portion configured to pierce a target tissue.

15. A method of forming an opening in a target tissue, the method comprising:
advancing a tissue perforation assembly that comprises a perforating member and a helical nosecone defining a nosecone channel, to a target tissue, wherein the helical nosecone comprises a helical slot spiraling around a tapering portion of the helical nosecone, the helical slot defining a plurality of helical turns;
forming, with the perforating member, a pilot puncture within the target tissue; and
advancing the helical nosecone in a screw-like motion through the pilot puncture.

16. The method of claim 15, wherein the advancing the helical nosecone through the pilot puncture is configured to expand the pilot puncture, to form a tissue opening within the target tissue.

17. The method of claim 15, wherein the perforating member is a guidewire extending through the nosecone channel.

18. The method of claim 15, wherein the perforating member is a tubular perforating member extending through the nosecone channel.

19. The method of claim 18, wherein the forming the pilot puncture comprises translating a distal end portion of the tubular perforating in a distal direction relative to the helical nosecone to pierce the target tissue to form the pilot puncture.

20. The method of claim 16, wherein the target tissue is a host leaflet of a host valvular structure, and wherein the tissue opening is a leaflet opening.

21. The method of claim 20, further comprising, subsequent to forming the leaflet opening:
positioning a guest prosthetic valve in a radially compressed state within the host valvular structure; and
radially expanding the guest prosthetic valve.
